(19) United States
(12) Patent Application Publication
(10) Pub. No.: US 2020/0009116 A1
(43) Pub. Date: Jan. 9, 2020

(54) ADMINISTRATION OF ANTIBIOTIC COMPOUNDS FOR THE TREATMENT OF STREPTOCOCCAL INFECTIONS FOR THE TREATMENT OF PSORIASIS

(71) Applicant: THE CENTRE FOR DIGESTIVE DISEASES, Five Duck (AU)

(72) Inventor: Thomas Julius BORODY, Five Dock (AU)

(21) Appl. No.: 16/474,028

(22) PCT Filed: Apr. 3, 2018
(86) PCT No.: PCT/IB2018/000441

§ 371 Date: Jun. 26, 2019

Related U.S. Application Data
(60) Provisional application No. 62/481,117, filed on Apr. 3, 2017.

(51) Int. Cl. A61K 31/438; A61P 31/04; A61K 9/00; A61K 31/498; A61K 31/7048; A61K 31/43; A61K 35/741; A61K 31/4164; A61K 38/14; A61K 31/437; A61P 17/06; A61K 31/546; A23L 33/10

(52) U.S. Cl. CPC A61K 31/438; A61P 31/04; A61K 9/0019; A61K 31/498; A61K 31/7048; A61K 31/43; A61K 2035/115; A61K 31/4164; A61K 38/14; A61K 31/437; A61P 17/06; A61K 31/546; A23L 33/10; A61K 35/741

(57) ABSTRACT

In alternative embodiments, provided are compositions, including therapeutic combinations of drugs, and methods of using them for e.g., treating, ameliorating and preventing various bacteria-induced conditions, disorders and infections in mammals, including genetically-predisposed and chronic disorders. In alternative embodiments, methods using the therapeutic combinations of drugs such as antibiotics, as provided herein, comprise or comprise use of medications, formulations and pharmaceuticals comprising active agent combinations as provided herein to e.g., treat, ameliorate, suppress or prevent a bacteria-induced condition, disorder or infection in a mammal. These therapeutic combinations of drugs, including medications, formulations and pharmaceuticals, are effective in a broad spectrum of disorders, including e.g., a skin or skin-related autoimmune disease or condition, e.g., a psoriasis.

### TECHNICAL FIELD

This invention generally relates to medicine, pharmacology and microbiology. In alternative embodiments, provided are compositions, including therapeutic combinations of drugs, and methods of using them for e.g., treating, ameliorating and preventing various bacteria-induced conditions, disorders and infections in mammals, including genetically-predisposed and chronic disorders. In alternative embodiments, methods using the therapeutic combinations of drugs such as antibiotics, as provided herein, are used to treat, ameliorate, suppress or prevent a bacteria-induced condition, disorder or infection in a mammal. These therapeutic combinations of drugs, including medications, formulations and pharmaceuticals, are effective in a broad spectrum of disorders, including e.g., a skin or skin-related autoimmune disease or condition, e.g., a psoriasis.

### BACKGROUND

Psoriasis is an autoimmune disease characterized by patches of abnormal skin, which may vary in severity from small and localized psoriasis lesions to complete body coverage. There is no cure for psoriasis. Current treatments that can help control the symptoms include steroid creams, omega-3 fatty acids (e.g., fish oil), barberry, vitamin D analogs (including vitamin D3 cream), topical retinoids, anthralin, calcineurin inhibitors, probiotics (e.g., L. acidophilus), light therapy (including ultraviolet light, sunlight, Goeckerman treatment), Excimer laser, salicylic acid, coal tar, moisturizers or aloe vera, and immune system suppressing medications such as methotrexate or amethopterin (optionally Trexall™, Rheumatrex™) or cyclosporine. Numerous triggers for psoriasis have been identified, including the use of antibiotics such as tetracycline and macrolides and penicillin, where all have been hypothesized to be risk factors for the onset of psoriasis (see e.g., Tsankov et al, J Am Acad Dermatol. 1988 October; 19(4):629-32; Kim et al, J Clin Aesthet Dermatol. 2010 January; 3(1): 32-38; Hong and Bernstein, Psoriasis Forum, Vol. 18, No. 1, (2012)).

### SUMMARY

In alternative embodiments, provided are therapeutic combinations, or compositions, for treating, ameliorating or preventing a bacterially-induced condition, infection or reaction in an individual in need thereof, or treating or ameliorating a bacterially-affected tissue, a bacterially-infected tissue,

wherein optionally the bacterially-induced condition, infection or reaction is a Streptococcal-induced condition, infection or reaction, or the bacterially-affected or a bacterially-infected tissue comprises a Streptococcal-affected or a Streptococcal-infected tissue,

and optionally the bacterially-induced condition, disease, infection or reaction is located in a tonsil, and optionally the tonsil is a palatine tonsil, an adenoid, and/or a tonsillar tissue at the base of the tongue or next to an eustachian tube,

wherein optionally the individual active agents (e.g., each individual antibiotic) of the therapeutic combination, or the composition are administered together (simultaneously), in a step-wise fashion, or lagging one by one to separate administration of each active agent,

and optionally the bacterially-induced condition, disease, infection or reaction is located in a tonsil, and optionally the tonsil is a palatine tonsil, an adenoid, and/or a tonsillar tissue at the base of the tongue or next to an eustachian tube,

wherein the therapeutic combination, or the composition, comprises:
(a) (1) clofazimine (optionally Lamprene™), an ansamycin, and amoxicillin (optionally Augmentin™, Amoxil™, Tyclav™, Synulox™, Dispermox™, Trimox™, Moxatag™),
   (2) clofazimine (optionally Lamprene™), an ansamycin (optionally rifabutin), amoxicillin (optionally Augmentin™, Amoxil™, Tyclav™, Synulox™, Dispermox™, Trimox™, Moxatag™) and clavulanic acid,
   (3) clofazimine (optionally Lamprene™) and an ansamycin,
   (4) clofazimine (optionally Lamprene™), an ansamycin, and a
combination of amoxicillin and clavulanic acid (Clavulin™),
wherein optionally the ansamycin is rifabutin (optionally Mycobutin™), rifampicin (also known as rifampin) (optionally Rifadin™), rifalazil (also known as KRM-1648 and AMI-1648) or a combination thereof,
and optionally:
(i) the clofazimine is administered or formulated for administration at a dose in the range of between about 10 to 800 mg/d (day), or between about 1.0 to 1000 mg/d (day)
(ii) the rifabutin is administered or formulated for administration at a dose in the range of between about 10 to 900 mg/d (day), or between about 5 to 1200 mg/d (day),
(iii) the rifabutin or rifampicin is administered or formulated for administration at a dose in the range of between about 10 to 900 mg/d (day), or between about 5 to 1200 mg/d (day),
(iv) the amoxicillin and/or clavulanic acid is/are individually administered or formulated for administration, or combined with another active agent, at a dose in the range of between about 10 to 2,000 mg/d (day), between about 5 to 4,000 mg/d (day), and optionally the another active agent is an amoxicillin (optionally Augmentin™, Amoxil™, Tyclav™, Synulox™, Dispermox™, Trimox™, Moxatag™), or
(v) any combination of (i) to (iv), or all of (i) to (iv);
(b) (1) rifabutin (optionally Mycobutin™), a macrolide antibiotic, and clofazimine (optionally Lamprene™),
   (2) rifabutin (optionally Mycobutin™) and a macrolide antibiotic,
   (3) a macrolide antibiotic and clofazimine (optionally Lamprene™),
   (4) rifabutin (optionally Mycobutin™) and clofazimine (optionally Lamprene™); or rifabutin (optionally Mycobutin™), clofazimine (optionally Lamprene™) and a macrolide antibiotic; or rifabutin (optionally Mycobutin™) and a macrolide antibiotic,
wherein optionally the macrolide antibiotic is clarithromycin (optionally Biaxin™), azithromycin (optionally Zithromax™, Azithrocin™), roxithromycin, erythromycin (optionally Eryc™, Erythrocin™) or a combination thereof,
wherein optionally the rifabutin is administered or formulated for administration at a dose in the range of between about 100 to 200 mg/dose, or for administration at a dosage of about between about 100 to 200 mg/d (day), or at a dosage of about 150 mg/d,
and optionally the clofazimine is administered or formulated for administration at a dose in the range of between about 25 to 150 mg/dose, or is administered or formulated for administration at a dose in the range of between about 25 to 150 mg/d,
and optionally the macrolide antibiotic (optionally clarithromycin) is administered or formulated for administration at a dose in the range of between about 50 to 300 mg/dose, or is administered or formulated for administration at a dose in the range of between about 50 to 300 mg/d, or at about 250 mg/d;

(c) (1) clofazimine (optionally Lamprene™) and ciprofloxacin (optionally Ciloxan™, Cipro™, Neofloxin™),
(2) ciprofloxacin (optionally Ciloxan™, Cipro™, Neofloxin™) and ansamycin, or
(3) clofazimine (optionally Lamprene™), ciprofloxacin (optionally Ciloxan™, Cipro™, Neofloxin™) and an ansamycin,
wherein optionally the ansamycin is rifabutin (optionally Mycobutin™), rifampicin (also known as rifampin) (optionally Rifadin™), rifalazil (also known as KRM-1648 and ANTI-1648) or a combination thereof,

(d) clofazimine (optionally Lamprene™), clindamycin (optionally Cleocin™, Dalacin™, Clinacin™) and clarithromycin (optionally Biaxin™);

(e) clofazimine (optionally Lamprene™), azithromycin (optionally Zithromax™, Azithrocin™) and cephalexin (also called cephalexin) (optionally Keflex™, Cepol™, Ceporex™);

(f) clofazimine (optionally Lamprene™), rifampicin (also known as rifampin) (optionally Rifadin™), and metronidazole (optionally Flagyl™, Metro™) or Tinidazole;

(g) rifampicin (also known as rifampin) (optionally Rifadin™), clofazimine (optionally Lamprene™), clarithromycin (optionally Biaxin™) and tinidazole (optionally Fasigyn™, Simplotan™, Tindamax™);

(h) amoxicillin (optionally Augmentin™, Amoxil™, Tyclav™, Synulox™, Dispermox™, Trimox™, Moxatag™), metronidazole (optionally Flagyl™, Metro™) and azithromycin (optionally Zithromax™, Azithrocin™);

(i) ciprofloxacin (optionally Ciloxan™, Cipro™, Neofloxin™), clofazimine (optionally Lamprene™) and amoxicillin (optionally Augmentin™, Amoxil™, Tyclav™, Synulox™, Dispermox™, Trimox™, Moxatag™),
wherein optionally the ciprofloxacin can be substituted with any quinolone or fluoroquinolone, wherein optionally the quinolone or the fluoroquinolone is moxifloxacin (optionally Avelox™, Avalox™, Avelon™, Vigamox™, Moxeza™), levofloxacin (optionally Levaquin™, Tavanic™, Iquix™), norfloxacin (optionally Noroxin™), of loxacin (optionally Floxin™, Ocuflox™) or gemifloxacin (optionally Factive™);

(j) metronidazole (optionally Flagyl™, Metro™, optionally an absorbable metronidazole) alone; or metronidazole (optionally Flagyl™, Metro™) and vancomycin (optionally Vancocin™, or a formulation as described in WO 2014085526 A1, optionally intravenously (IV) administered vancomycin (formulated for IV administration); or metronidazole (optionally Flagyl™, Metro™) and rifaximin (optionally Xifaxan™, Xifaxanta™, Normix™); or metronidazole (optionally Flagyl™, Metro™, optionally an absorbable metronidazole), vancomycin (optionally Vancocin™, or a formulation as described in WO 2014085526 A1, optionally intravenously (IV) administered vancomycin (formulated for IV administration)) and rifaximin (optionally Xifaxan™, Xifaxanta™, Normix™);

(k) a bactericidal lipoglycopeptide and a cephalosporin (optionally vancomycin (optionally Vancocin™, or a formulation as described in WO 2014085526 A1, optionally intravenously (IV) administered vancomycin (formulated for IV administration) and ceftriaxone (optionally Rocephin™, Epicephin™)),
wherein optionally the cephalosporin is ceftaroline fosamil (optionally Teflaro™, Zinforo™), cephacetrile, cefadroxyl (optionally Duricef™), cephalexin (also called cephalexin) (optionally Keflex™, Cepol™, Ceporex™); cephaloglycin, cephalonium, cephaloridine, cephalothin (optionally Keflin™), cephapirin (optionally Cefadryl™), cefatrizine, cefazaflur, cefazedone, cefazolin (or cephazolin; optionally Ancef™, Kefzol™), cefradine (or cephradine; optionally Velosef™), cefaclor (optionally Ceclor™, Distaclor™, Keflor™, Raniclor™), cefonicid (optionally Monocid™), cefprozil (or cefproxil, optionally Cefzil™), cefuroxime (optionally Zefu™, Zinnat™, Zinacef™, Ceftin™, Biofuroksym™, Xorimax™), loracarbef (optionally Lorabid™), cefmetazole (optionally Zefazone™), cefotetan (optionally Cefotan™), cefoxitin (optionally Mefoxin™), cefotiam (optionally Pansporin™), cefdinir (optionally Sefdin™, Zinir™, Omnicef™, Kefnir™), cefixime (optionally Fixx™, Zifi™, Suprax™), cefotaxime (optionally Claforan™), cefovecin (optionally Convenia™), cefpodoxime (optionally Vantin™, PECEF™, Simplicef™), cefteram, ceftamere (optionally Enshort™), ceftibuten (optionally Cedax™), ceftiofur (optionally Naxcel™, Excenel™), ceftiolene, ceftizoxime (optionally Cefizox™), ceftriaxone (optionally Rocephin™, Epicephin™), cefoperazone (optionally Cefobid™), ceftazidime (optionally Meezat™, Fortum™, Fortaz™), cefepime (optionagarglly Maxipime™, Voco™), cefpirome (optionally Cefrom™), or flomoxef,
wherein optionally the bactericidal lipoglycopeptide is telavancin (optionally Vibativ™), and optionally the lipopeptide antibiotic is daptomycin (Cubicin™), and optionally the glycopeptide is bleomycin (Blenoxane™), teicoplanin (Targocid™), or a vancomycin (optionally Vancocin™, or a formulation as described in WO 2014085526 A1, optionally intravenously (IV) administered vancomycin (formulated for IV administration); and/or

(l) a selection or combination of at least one, two, three, four, five, six or seven or more of any antibiotic or antibiotics selected from:

amikacin (optionally Amikin™);

an aminoglycoside,
wherein optionally the aminoglycoside is amikacin (optionally Amikin), tobramycin (optionally Tobrex™), dibekacin, kanamycin, gentamycin (optionally Cidomycin™, Septopal™, Genticyn™, Garamycin™), sisomicin (also known as ensamycin) (optionally bactoCeaze™), neomycin (optionally Neo-rx™), netilmicin, paromomycin (optionally Catenulin™, Aminosidine™) or streptomycin;

amoxicillin (optionally Augmentin™, Amoxil™, Tyclav™, Synulox™, Dispermox™, Trimox™, Moxatag™);

an ansamycin,
wherein optionally the ansamycin is rifabutin (optionally Mycobutin™), rifampicin (also known as rifampin) (optionally Rifadin™), rifalazil (also known as KRM-1648 and ANTI-1648) or a combination thereof; azithromycin (optionally Zithromax™, Azithrocin™);

a beta-lactam antibiotic,
wherein optionally the beta-lactam antibiotic is a penicillin a carbapenem, a cephalosporin, or a monobactam,
and optionally the penicillin is benzylpenicillin (also known as penicillin G) (optionally Pfizerpen™), benzathine benzylpenicillin (also known as benzathine penicillin G), phenoxymethylpenicillin (also known as penicillin V) (optionally Veetids™), or procaine benzylpenicillin (also known as penicillin G procaine) (optionally Bicillin C-R™),
and optionally the carbapenem is imipenem (optionally Primaxin™), meropenem (optionally Merrem™), ertapenem (optionally Invanz™), doripenem (optionally Finibax™, Doribax™), panipenem (also called betamipron), biapenem, razupenem (optionally PTZ-601™), tebipenem (optionaly Orapenem™),
and optionally the monobactam is aztreonam (optionally Azactam™, Cayston™), tigemonam, nocardicin A, tabtoxin,
and optionally the cephalosporin is ceftaroline fosamil (optionally Teflaro™, Zinforo™), cephacetrile, cefadroxyl (optionally Duricef™), cephalexin (also called cephalexin) (optionally Keflex™, Cepol™, Ceporex™); cephaloglycin, cephalonium, cephaloridine, cephalothin (optionally Keflin™), cephapirin (optionally Cefadryl™), cefatrizine, cefazaflur, cefazedone, cefazolin (or cephazolin; optionally Ancef™, Kefzol™), cefradine (or cephradine; optionally Velosef™), cefaclor (optionally Ceclor™, Distaclor™, Keflor™, Raniclor™), cefonicid (optionally Monocid™), cefprozil (or cefproxil, optionally Cefzil™) cefuroxime (optionally Zefu™, Zinnat™, Zinacef™, Ceftin™, Biofuroksym™, Xorimax™), loracarbef (optionally Lorabid™), cefmetazole (optionally Zefazone™), cefotetan (optionally Cefotan™), cefoxitin (optionally Mefoxin™), cefotiam (optionally Pansporin™), cefdinir (optionally Sefdin™, Zinir™, Omnicef™, Kefnir™), cefixime (optionally Fixx™, Zifi™, Suprax™), cefotaxime (optionally Claforan™), cefovecin (optionally Convenia™), cefpodoxime (optionally Vantin™, PECEF™, Simplicef™), cefteram, ceftamere (optionally Enshort™), ceftibuten (optionally Cedax™), ceftiofur (optionally Naxcel™, Excenel™), ceftiolene, ceftizoxime (optionally Cefizox™), ceftriaxone (optionally Rocephin™, Epicephin™), cefoperazone (optionally Cefobid™), ceftazidime (optionally Meezat™, Fortum™, Fortaz™), cefepime (optionagarglly Maxipime™, Voco™), cefpirome (optionally Cefrom™), or flomoxef;

clarithromycin (optionally Biaxin™);

clavulanic acid;

clofazimine (optionally Lamprene™);

ethambutol (optionally Myambutol™, Etibi™, Servambutol™);

fosomycin (optionally Monurol™, Monuril™);

a lincosamide,
wherein optionally the lincosamide is lincomycin, pirlimycin, or clindamycin (optionally Cleocin™, Dalacin™, Clinacin™);

a bactericidal lipoglycopeptide, a glycopeptide, or a lipopeptide antibiotic,
wherein optionally the bactericidal lipoglycopeptide is telavancin (optionally Vibativ™), and optionally the lipopeptide antibiotic is daptomycin (Cubicin™), and optionally the glycopeptide is bleomycin (Blenoxane™), teicoplanin (Targocid™), or a vancomycin (optionally Vancocin™, or a formulation as described in WO 2014085526 A1, optionally intravenously (IV) administered vancomycin (formulated for IV administration);

a nystatin (optionally Mycostatin™, Nystop™),

a oxazolidinone,
wherein optionally the oxazolidinone is linezolid (optionally Zyvox™, Zyvoxid™), posizolid, tedizolid (optionally Sivextro™), radezolid (optionally RX-1741™) or cycloserine (optionally Seromycin™),

a quinolone or a fluoroquinolone,
wherein optionally the quinolone or the fluoroquinolone is moxifloxacin (optionally Avelox™, Avalox™, Avelon™, Vigamox™, Moxeza™), ciprofloxacin (optionally Ciloxan™, Cipro™, Neofloxin™), levofloxacin (optionally Levaquin™, Tavanic™, Iquix™), norfloxacin (optionally Noroxin™), ofloxacin (optionally Floxin™, Ocuflox™) or gemifloxacin (optionally Factive™);

a macrolide antibiotic,
wherein optionally the macrolide antibiotic is a ketolide antibiotic, clarithromycin (optionally Biaxin™), azithromycin (optionally Zithromax™, Azithrocin™), roxithromycin, erythromycin (optionally Eryc™, Erythrocin™),
and optionally the ketolide antibiotic is telithromycin (Ketek™);

metronidazole (optionally Flagyl™, Metro™, optionally an absorbable metronidazole);

a polyketide antibiotic (optionally anthracimycin, geldanamycin, doxycycline (optionally Doryx™, Doxyhexal™, Doxylin™), erythromycin (optionally Eryc™, Erythrocin™);

rifampicin (also known as rifampin) (optionally Rifadin™),

rifaximin (optionally Xifaxan™, Xifaxanta™, Normix™),

a streptogramin,
wherein optionally the streptogramin is pristinamycin (optionally Pyostacine™), quinupristin, dalfopristin, or both Quinupristin and dalfopristin (optionally Synercid™);

a sulphonamide,
wherein optionally the sulphonamide is hydrochlorothiazide (optionally Apo-hydro™), furosemide (optionally Lasix™) or sulfamethoxazole (optionally Gantanol™);

tinidazole (optionally Fasigyn™, Simplotan™, Tindamax™);

tigecycline (optionally Tygacil™); and

trimethoprim (optionally Proloprim™, Monotrim™, Triprim™).

In alternative embodiments, the therapeutic drug combination or composition, or individual elements of the therapeutic drug combination or composition, is formulated for administration once a day, b.i.d. (twice a day) or t.i.d, (three times a day), or weekly, or biweekly, or monthly. In alternative embodiments, the therapeutic combination, or the composition, or individual elements of the therapeutic drug combination or composition, are formulated for or formulated as: administration intravenously, topically, orally, by gargling, by inhalation, by infusion, by injection, by inhalation, intraperitoneally, intramuscularly, subcutaneously, intra-aurally, for intra-articular administration, for intra-mammary administration, for topical administration or for absorption through epithelial or mucocutaneous linings, conventional or PEGylated liposomal formulations. In alternative embodiments, the therapeutic combination, or the composition, or individual elements of the therapeutic drug combination or composition, is formulated for intermittent or alternated cycling of the drug or active agent or component, and optionally the intermittent or alternated cycling comprises administration weekly, bi-monthly, monthly or quarterly.

In alternative embodiments, provided are pharmaceutical compositions or formulations comprising the therapeutic combination, or a composition, as provided herein. In alternative embodiments, the pharmaceutical compositions or the formulations further comprise a pharmaceutically acceptable excipient.

In alternative embodiments, the pharmaceutical composition or formulation is formulated or manufactured as a candy, a lollipop (or lollie, pop or sucker), a disposable wafer, strip or patch, a feed, a food, a food or feed concentrate, a pellet, a lozenge, a liquid, a lotion, an implant, a nanoparticle, an elixir, an aerosol, a spray, an inhalant, a powder, a tablet, a pill, a capsule, a gel, a geltab, a nanosuspension, a microparticle or a nanoparticle, a patch, a microgel, a liposome, or a suppository, and optionally the pharmaceutical composition or the formulation further comprises a probiotic, optionally a probiotic lozenge.

In alternative embodiments, provided are devices (e.g., medical devices), implants (e.g., neck, mouth, throat implants), a prosthesis, a stent, a catheter, a spray or an aerosol device, an inhaler, a nebulizer, an atomizing device, or a neck, pharyngeal or oral implant: comprising a therapeutic combination, or a composition, as provided herein, or a pharmaceutical composition or a formulation as provided herein.

In alternative embodiments, provided are methods for treating, ameliorating or preventing a bacterially-induced disease, condition, infection or reaction in an individual in need thereof; or, applying or administering to a bacterially-affected or a bacterially-infected tissue, wherein optionally the bacterially-induced condition, infection or reaction is a Streptococcal-induced condition, infection or reaction, or the bacterially-affected or a bacterially-infected tissue comprises a Streptococcal-affected or a Streptococcal-infected tissue, the method comprising an individual in need thereof a therapeutic combination or a composition as provided herein, or a pharmaceutical composition or a formulation as provided herein, or a device, implant, prosthesis, stent or catheter as provided herein, wherein optionally the bacterially-induced disease, condition, infection or reaction in the individual in need thereof is an autoimmune disease, reaction or condition, wherein optionally the autoimmune disease, reaction or condition is:
(1) a skin or skin-related autoimmune disease or condition,
   and optionally the skin or skin-related autoimmune disease or condition is a psoriasis, optionally a plaque-, guttate-, inverse-, pustular- or erythrodermic-type psoriasis; or a Pustulosis Palmaris et Plantaris (PPP) or a Palmoplantar pustulosis,
(2) a Pediatric Autoimmune Neuropsychiatric Disorder (optionally a Pediatric Autoimmune Neuropsychiatric Disorder Associated with a Streptococcal Infection (PANDAS)),
(3) rheumatic fever or rheumatic heart disease,
(4) reactive arthritis, or
(5) acute glomerulonephritis,

and optionally the Streptococcal organism comprises a Group A, B, C, D, F, G or H Streptoccocus, a Streptococcus pneumoniae or a Streptoccocus viridans, wherein optionally the Group A Streptococcal organism is a Streptoccocus pyogenes or Streptococcal pyoderma, and optionally the Group B Streptococcal organism is a Streptococcus agalactiae, and optionally the Group F Streptococcal organism is a Streptococcus anginosus (S. anginosus) or a S. salivarius,

and optionally the therapeutic combination or the compositions or the pharmaceutical composition or the formulation, or individual elements of the therapeutic combination or the composition or the pharmaceutical composition or the formulation, are administered separately or together, or at the same time, or in synchrony, or by chrono-dosing, or one of the therapeutic agents or drugs is administered before another of the therapeutic agents or drugs,

and optionally when three or more therapeutic combinations or compositions are to be administered to an individual in need thereof during a course of treatment, or when three or more individual active agents (optionally antibiotics) are to be administered to an individual in need thereof during a course of treatment, then two of the three or more of the therapeutic combinations or compositions or three or more of the active agents (optionally antibiotics) are first administered, optionally in one unit dosage form (optionally a tablet, capsule, spray, aerosol or lozenge), and after a period of time (optionally between one week and one month, or between one month and one year) the third or more of the therapeutic combinations or compositions or of the active agents (optionally antibiotics) are added to the treatment regimen to the individual in need thereof,

and optionally the therapeutic combination comprising the three active agents rifabutin, clofazimine and macrolide antibiotic (optionally clarithromycin) is administered to the individual in need thereof, and treatment is initiated by first administering two of the three active agents rifabutin, clofazimine and macrolide antibiotic, and after a period of time (optionally between one week and one month, or between one month and one year) the third active agent antibiotic is added to the treatment regimen to the individual in need thereof, and optionally the first two administered active agents are: rifabutin and clofazimine; rifabutin and macrolide antibiotic; or, clofazimine and macrolide antibiotic,

and optionally when the therapeutic combination comprises the three active agents rifabutin, clofazimine and macrolide antibiotic (optionally clarithromycin) is administered to the individual in need thereof, the rifabutin (optionally Mycobutin™) is administered first at a dosage of between about 100 to 200 mg/day, or at about 150 mg/day (d) (optionally administered in the morning), and about 2 to 4 weeks later the macrolide antibiotic (optionally clarithromycin) is added administered at a dosage of between about 200 to 300 mg/d, or at about 250 mg/d (optionally administered in the morning), and about 2 to 4 weeks later the clofazimine is added at a dose in the range of between about 25 to 150 mg/dose, or 50 to 100 mg/d, or at 75 mg/d (optionally administered in the morning),

and optionally at about 1 week to 2 months, or about 2 to 4 weeks, after commencing of administration of all three rifabutin, clofazimine and macrolide antibiotic, begin administering the same range dosages twice a day, or bid (rifabutin at a dosage of between about 100 to 200 mg/day, the macrolide antibiotic (optionally clarithromycin) is added administered at a dosage of between about 200 to 300 mg/d, the clofazimine is added at a dose in the range of between about 25 to 150 mg/dose, or 50 to 100 mg/d), and optionally the bid dosages of all three rifabutin, clofazimine and macrolide antibiotic remain the same as with once a day dosaging, or the clofazimine bid dosage is set at about 75 mg/d;

and optionally at about 1 week to 2 months, or after about 2 to 4 weeks, after commencing the bid dosaging, increasing the dosage bid of rifabutin and macrolide antibiotic, optionally increasing the dosage bid of rifabutin and macrolide antibiotic to: about 300 mg bid for rifabutin and/or about 500 mg macrolide antibiotic bid, and optionally the clofazimine bid dosage remains the same, or in the range of between about 25 to 150 mg/dose, or 50 to 100 mg/d, or at 75 mg/d,

and optionally the therapeutic combination comprising the three active agents clofazimine, an ansamycin, and amoxicillin is administered to the individual in need thereof, and treatment is initiated by first administering two of the three active agents clofazimine, an ansamycin, and amoxicillin, and after a period of time (optionally between one week and one month, or between one month and one year) the third active agent antibiotic is added to the treatment regimen to the individual in need thereof, and optionally the first two administered active agents are: clofazimine and an ansamycin; an ansamycin and amoxicillin; or clofazimine and amoxicillin,

and optionally the therapeutic combination or the compositions or the pharmaceutical composition or the formulation, or individual elements of the therapeutic combination or the composition or the pharmaceutical composition or the formulation, are formulated for administration intravenously (IV), parenterally, nasally, topically or locally, orally, or by liposome, implant or vessel-targeted nanoparticle delivery,

and optionally further comprises administering a lozenge, a tablet, a liquid, an aerosol, a spray, a geltab or a gel,
wherein optionally the lozenge is a probiotic lozenge, or the tablet, liquid, aerosol, spray, geltab or gel comprises a probiotic,
and optionally the lozenge is a Blis K12 Throat Guard™ or a Blis K12 Throat Guard Boost™ lozenge,
and optionally the probiotic, liquid or gel comprises a non-pathogenic bacterial strain, wherein optionally the non-pathogenic (or attenuated) bacterial strain is or comprises a Streptococcus, optionally a Streptococcus salivarius, optionally Streptococcus salivarius K12,
and optionally the lozenge, liquid or gel is administered after the antibiotics have been administered (after termination of the antibiotic administration regimen),
and optionally the non-pathogenic (or attenuated) bacteria, optionally a Streptococcus strain, partially, substantially or completely replaces (e.g., between about 70% to 90% replaces, between about 80% to 95% replaces, or between about 90% to 99% replaces, or replaces 100% of) a pathogenic bacteria in an infected tissue, wherein optionally the infected tissue is an adenoid or a tonsil,
and optionally killed pathogenic Streptococci, or non-pathogenic Streptococci, may be used as an oral vaccine to stimulate the immune system to control and kill an infecting Streptococcus in a tonsillar tissue and/or an adenoid.

In alternative embodiments, provided are methods wherein the level of dosing continues daily (or optionally after the second or third daily dose, administered less frequently) for prolonged periods (optionally between one week and ten years), and optionally the dosing can be divided into an induction therapy for about 7 days or 5 days to two weeks (or 1, 2, 3, 4, 5, 6, 7, 8, 9, 10, 11, 12, 13, 14 or 15 or more days), and optionally for up to about four, five or six months, or up to between one month and two years, or two months and one year, with the dose being reduced (optionally halved or quartered, or reduced dosage from between about 10% to about 90% dose reduction) thereafter (optionally after five to 12 days, or after a week, or after two weeks) to a reduced maintenance therapy.

In alternative embodiments, methods as provided herein further comprise: before, during or after the applying or administering the therapeutic combination or the compositions or the pharmaceutical composition or the formulation, or individual elements of the therapeutic combination or the composition or the pharmaceutical composition or the formulation:

(a) a surgical procedure comprising a tonsillectomy or adenoid removal, wherein optionally the tonsillectomy is a palatine tonsillectomy and/or an adenoid tonsillectomy, or the method comprises complete or partial removal, freezing, coagulation or ablation of one, any of or all of: a palatine tonsil, an adenoid, and/or a tonsillar tissue at the base of the tongue or next to an eustachian tube; and/or

(b) an immunization of an individual in need thereof against a Streptococcal organism,

wherein optionally the immunization comprises immunizing with an attenuated or a killed pathogenic bacteria, optionally an attenuated or a killed Streptococci, optionally an attenuated or a killed pathogenic Streptococci which has been cultured from a tonsil or a removed tonsil of a patient with a psoriasis,

and optionally the immunization comprises immunizing with a polyvalent immunizing agent,

and optionally the immunization comprises immunizing with an immunizing agent that can either be ingested or used to immunize by injecting into and/or under the skin,

and optionally the immunization is designed to, or results in, an immunized individual developing antibodies and/or a T-cell immunity against a Streptococci (optionally a lymph node Streptococci, optionally a tonsillar lymph node Streptococci),

and optionally the immunization against a Streptococcal organism further comprises administration of an immune-modulator to the individual in need thereof,
and optionally the immune-modulator comprises alefacept (optionally Amevive™), efalizumab (optionally Raptiva™), etanercept (optionally Enbrel™), ixekinumab (optionally Talz™), golimumab (optionally Simponi™), guselkumab (optionally Tremfya™), cetrolizumab-pegol (optionally Cimzia™), tildrakizumab (optionally Ilumya™), abatacept (optionally Orencia™), an anti-TNF infusion or product (e.g., adalimumab (optionally Humira™, Exemptia™) or infliximab (optionally Remicade™, Remsima™, Inflectra™)), anti-IL 12/23 (optionally ustekinumab, or Stelara™), anti-IL 23, anti-IL 17F, or anti-IL 17A (optionally secukinumab, or Cosentyx™) products, prednisone (optionally Deltasone™, Liquid Pred™, Orasone™, Adasone™, Deltacortisone™, Prednisonum™), cyclosporine or ciclosporine (optionally Neoral™, Sandimmune™), mercaptopurine or 6-MP (6-mercaptopurine) (optionally Purinethol™), methotrexate or amethopterin (optionally Trexall™, Rheumatrex™), tacrolimus (fujimycin) (optionally Prograf™ Advagraf™, Protopic™), ascomycin or immunomycin, rapamycin or sirolimus (optionally Rapamune™), sulfasalazine (optionally Azulfidine™, Salazopyrin™, Sulazine™), a steroid (e.g., a corticosteroid), azathioprine (optionally Azasan™, Imuran™), or a combination thereof,
and optionally administration of the immunomodulator occurs concurrently with the antibiotic administration, optionally administration of the immunomodulator commences as antibiotic treatment commences, or optionally administration of the immunomodulator commences when and if the antibiotic treatment requires support before reaching therapeutically adequate suppression or eradication of the targeted bacteria in the individual (before reaching therapeutically adequate suppression or eradication of the bacteria causing the bacterially-induced condition, infection or reaction in an individual in need thereof, or before reaching therapeutically adequate suppression or eradication of the bacteria infecting the bacterially-affected or a bacterially-infected tissue; and/or

(c) administering a lozenge, a tablet, a liquid, an aerosol, a spray, a geltab or a gel,

wherein optionally the lozenge is a probiotic lozenge, or the tablet, liquid, aerosol, spray, geltab or gel comprises a probiotic,

and optionally the lozenge is a Blis K12 Throat Guard™ or a Blis K12 Throat Guard Boost™ lozenge,

and optionally the probiotic, liquid or gel comprises a non-pathogenic bacterial strain, wherein optionally the non-pathogenic (or attenuated) bacterial strain is or comprises a Streptococcus, optionally a Streptococcus salivarius, optionally Streptococcus salivarius K12,

and optionally the lozenge, liquid or gel is administered after the antibiotics have been administered (after termination of the antibiotic administration regimen),

and optionally the non-pathogenic (or attenuated) bacteria, optionally a Streptococcus strain, partially, substantially or completely replaces (e.g., between about 70% to 90% replaces, between about 80% to 95% replaces, or between about 90% to 99% replaces, or replaces 100%) a pathogenic bacteria in an infected tissue, wherein optionally the infected tissue is an adenoid or a tonsil,

and optionally antibiotic treatment can be concurrent with a Streptococcus salivarius formulation such as lozenges, liquids, gels, and the like; and optionally antibiotic treatment can be concurrent with as oral vaccination with a killed or attenuated pathogenic strains. Treatment can then be finalised with a tonsillectomy.

In alternative embodiments, provided are uses of a therapeutic combination, or a composition, for (or said use being in) the manufacture of a medicament for treating, ameliorating or preventing a bacterially-induced condition, infection or reaction, optionally a Streptococcal-induced condition, infection or reaction, or a bacterial infection, optionally a Streptococcal-induced or Streptococcal infection, in an individual in need thereof, wherein the therapeutic combination, or the composition comprises a therapeutic combination, or a composition as provided herein.

In alternative embodiments, provided are formulations or therapeutic combinations for use in a method of treating, ameliorating or preventing a bacterially-induced condition or reaction, optionally a Streptococcal-induced condition, infection or reaction, or a bacterial infection, optionally a Streptococcal-induced or Streptococcal infection, in an individual in need thereof, wherein the formulation or a therapeutic combination comprises a therapeutic combination, or a composition as provided herein, and the method comprises a method as provided herein.

The details of one or more embodiments of the invention are set forth in the description below. Other features, objects, and advantages of the invention will be apparent from the description and from the claims.

All publications, patents, patent applications cited herein are hereby expressly incorporated by reference for all purposes.

Reference will now be made in detail to various exemplary embodiments of the invention. The following detailed description is provided to give the reader a better understanding of certain details of aspects and embodiments of the invention, and should not be interpreted as a limitation on the scope of the invention.

### DETAILED DESCRIPTION

In alternative embodiments, provided are therapeutic combinations, compositions, for treating, ameliorating or preventing a bacterially-induced condition, infection or reaction in an individual (including a human or a non-human animal) in need thereof, or treating or ameliorating a bacterially-affected or a bacterially-infected tissue in an individual. In alternative embodiments, the bacterially-induced condition, infection or reaction is a Streptococcal-induced condition, infection or reaction, or the bacterially-affected or a bacterially-infected tissue comprises a Streptococcal-affected or a Streptococcal-infected tissue. In alternative embodiments, provided are therapeutic combinations, compositions, for treating, ameliorating or preventing an autoimmune disease or condition, or a psoriasis.

In alternative embodiments, provided are devices such as medical devices, implants such as breast implants, prostheses, stents, catheters, pins, plates and the like comprising a therapeutic combination of antibiotics, or antibiotics and additional drugs, as provided herein.

While the invention is not limited to any particular mechanism of action, from the point of causality for psoriasis, a chronic Streptococcal throat infection affecting the tonsils is likely. T cells from patients with psoriasis show increased responses to homologous peptides from Streptococcal M proteins and human epidermal keratins, thus, it is possible that psoriasis may occur as a consequence of T cells cross-reacting with epitopes from Streptococcal M proteins and human keratins. The palatine tonsils might play a major role in psoriasis as they are a common site for Streptococcal infections.

### Formulations and Dosages

In alternative embodiments, compositions, including therapeutic combinations, as provided herein, including pharmaceuticals such as antibiotics, formulations, and compositions used to practice the methods as provided herein, comprise specific active agents as provided herein and their polymorphic forms or analogs thereof, or equivalents thereof; and these can be formulated in particular delivery forms, e.g., for oral delivery, for example, in alternative embodiments, for delivery of active agent, e.g., antibiotics, to the throat or to tonsillar tissues. In alternative embodiments, therapeutic combinations as provided herein are formulated for pediatric use, e.g., for specially applicability to children, and/or for long-term use, particularly for children, for example, as lozenges, dissolvable wafers or patches, lollies (e.g., lollypops, “pops” or suckers), candies, gums, aerosols and sprays, where therapeutic combinations of antibiotics (or just one or more of a therapeutic combination) as provided herein are delivered orally to the individual in need thereof, e.g., child or an adult, by use of the lozenges, dissolvable wafers or patches, lollies (e.g., lollypops, “pops” or suckers), candies, gums, aerosols and sprays.

In alternative embodiments, compositions as provided herein, including therapeutic combinations (or a single member of a therapeutic combination), and compositions used to practice the methods of the invention, can take the form of (e.g., to be formulated as, or to be delivered orally by use of) a capsule, a geltab, a pill, a dissolvable wafer or patch, a tablet (e.g., an orally disintegrating sublingual tablet or buccal tablet or wafer), a chewable sweet, a sweet, a lolly (e.g., lollypops, “pops” or suckers), a gum, a lozenge, a candy, a smoothy, a jelly, an ice, ice cream, gelato, yogurt or a drink. In alternative embodiments, compositions as provided herein, including therapeutic combinations (or a single member of a therapeutic combination), and compositions used to practice the methods of the invention, can be formulated as, or be in the form of, ice (e.g., an ice cube or shaving) to be added to a liquid, gel or any food to be given to an individual in need thereon, e.g., a child or an adult.

In alternative embodiments, a delivery vehicle or form contains or carries the formulation components, e.g., a yogurt or a drink, and the delivery vehicle or form is designed such that a solid, gel or liquid component of an antibiotic or a therapeutic combination as provided herein, is kept without degradation inside a separate sealed space which can be emptied into the delivery form or vehicle, e.g., drink or yogurt. For example, by using a twist top or equivalent the contents of the separate container (e.g., the antibiotic or the therapeutic combination as provided herein) will be released into the delivery formulation, e.g., the yogurt or drink. For example, by twisting a twist top or equivalent a solid form (e.g., granules) or gel or liquid form of an active agent (e.g., an antibiotic or combinations of antibiotics as provided herein), are released into the drink, or yogurt or the like, which then dissolve, disperse or intermix with the drink, or yogurt or the like and are eaten or drunk by the individual, e.g., a child.

In alternative embodiments, a product, a delivery form or formulation as provided herein, for example, is a flavoured chewable tablet (e.g., an orally disintegrating sublingual tablet or buccal tablet or wafer), lolly, pop, sucker, lozenge, sweet or candy which an individual, e.g., a child, is directed to take, e.g., more than once a day, e.g., twice or three times daily, to maintain a desired therapeutic result.

In alternative embodiments, doses (unit dosage forms) of active agents such as antibiotics as provided herein, are administered to individuals in need thereof, e.g., individuals with psoriasis. In alternative embodiments, antibiotics, polymorphic forms or analogs thereof, or equivalents thereof, are administered at unit dosages of from between about 10 mg through or to about 10 gms, or between about 100 mg and 500 mgm, or at 10, 20, 30, 40, 50, 75, 100, 200, 300, 400, 500 or 1000 mgm. Dosages can be adjusted depending on the combination of active agents used, particularly when using two or three or more combination drugs, e.g., combinations of antibiotics.

For example, in alternative embodiments, therapeutic combinations used to practice the compositions or methods of the invention include: a selection or combination of at least one, two, three, four, five, six or seven or more of any antibiotic or antibiotic combinations selected from: amikacin (optionally Amikin™); an aminoglycoside, wherein optionally the aminoglycoside is amikacin (optionally Amikin), tobramycin (optionally Tobrex™), dibekacin, kanamycin, gentamycin (optionally Cidomycin™, Septopal™, Genticyn™, Garamycin™), sisomicin (also known as ensamycin) (optionally bactoCeaze™), neomycin (optionally Neo-rx™), netilmicin, paromomycin (optionally Catenulin™, Aminosidine™) or streptomycin; amoxicillin (optionally Augmentin™, Amoxil™, Tyclav™, Synulox™, Dispermox™, Trimox™, Moxatag™); an ansamycin, wherein optionally the ansamycin is rifabutin (optionally Mycobutin™), rifampicin (also known as rifampin) (optionally Rifadin™), rifalazil (also known as KRM-1648 and AMI-1648) or a combination thereof; azithromycin (optionally Zithromax™, Azithrocin™); a beta-lactam antibiotic, wherein optionally the beta-lactam antibiotic is a penicillin a carbapenem, a cephalosporin, or a monobactam, and optionally the penicillin is benzylpenicillin (also known as penicillin G) (optionally Pfizerpen™), benzathine benzylpenicillin (also known as benzathine penicillin G), phenoxymethylpenicillin (also known as penicillin V) (optionally Veetids™), or procaine benzylpenicillin (also known as penicillin G procaine) (optionally Bicillin C-R™), and optionally the carbapenem is imipenem (optionally Primaxin™), meropenem (optionally Merrem™), ertapenem (optionally Invanz™), doripenem (optionally Finibax™, Doribax™), panipenem (also called betamipron), biapenem, razupenem (optionally PTZ-601™), tebipenem (optionaly Orapenem™), and optionally the monobactam is aztreonam (optionally Azactam™, Cayston™), tigemonam, nocardicin A, tabtoxin, and optionally the cephalosporin is ceftaroline fosamil (optionally Teflaro™, Zinforo™), cephacetrile, cefadroxyl (optionally Duricef™), cephalexin (also called cephalexin) (optionally Keflex™, Cepol™, Ceporex™); cephaloglycin, cephalonium, cephaloridine, cephalothin (optionally Keflin™), cephapirin (optionally Cefadryl™), cefatrizine, cefazaflur, cefazedone, cefazolin (or cephazolin; optionally Ancef™, Kefzol™), cefradine (or cephradine; optionally Velosef™), cefaclor (optionally Ceclor™, Distaclor™, Keflor™, Raniclor™), cefonicid (optionally Monocid™), cefprozil (or cefproxil, optionally Cefzil™), cefuroxime (optionally Zefu™, Zinnat™, Zinacef™, Ceftin™, Biofuroksym™, Xorimax™), loracarbef (optionally Lorabid™), cefmetazole (optionally Zefazone™), cefotetan (optionally Cefotan™), cefoxitin (optionally Mefoxin™), cefotiam (optionally Pansporin™), cefdinir (optionally Sefdin™, Zinir™, Omnicef™, Kefnir™), cefixime (optionally Fixx™, Zifi™, Suprax™), cefotaxime (optionally Claforan™), cefovecin (optionally Convenia™), cefpodoxime (optionally Vantin™, PECEF™, Simplicef™), cefteram, ceftamere (optionally Enshort™), ceftibuten (optionally Cedax™), ceftiofur (optionally Naxcel™, Excenel™), ceftiolene, ceftizoxime (optionally Cefizox™), ceftriaxone (optionally Rocephin™, Epicephin™), cefoperazone (optionally Cefobid™), ceftazidime (optionally Meezat™, Fortum™, Fortaz™), cefepime (optionally Maxipime™, Voco™), cefpirome (optionally Cefrom™), or flomoxef; clarithromycin (optionally Biaxin™); clavulanic acid; clofazimine (optionally Lamprene™); ethambutol (optionally Myambutol™, Etibi™, Servambutol™); fosomycin (optionally Monurol™, Monuril™); a lincosamide, wherein optionally the lincosamide is lincomycin, pirlimycin, or clindamycin (optionally Cleocin™, Dalacin™, Clinacin™); a bactericidal lipoglycopeptide or a lipopeptide antibiotic, wherein optionally the bactericidal lipoglycopeptide is telavancin (optionally Vibativ™), and optionally the lipopeptide antibiotic is daptomycin (Cubicin™); a oxazolidinone, wherein optionally the oxazolidinone is linezolid (optionally Zyvox™, Zyvoxid™), posizolid, tedizolid (optionally Sivextro™), radezolid (optionally RX-1741™) or cycloserine (optionally Seromycin™), a quinolone or a fluoroquinolone, wherein optionally the quinolone or the fluoroquinolone is moxifloxacin (optionally Avelox™, Avalox™, Avelon™, Vigamox™, Moxeza™), ciprofloxacin (optionally Ciloxan™, Cipro™, Neofloxin™), levofloxacin (optionally Levaquin™, Tavanic™, Iquix™), norfloxacin (optionally Noroxin™) ofloxacin (optionally Floxin™, Ocuflox™) or gemifloxacin (optionally Factive™); a macrolide antibiotic, wherein optionally the macrolide antibiotic is a ketolide antibiotic, clarithromycin (optionally Biaxin™), azithromycin (optionally Zithromax™, Azithrocin™), roxithromycin, erythromycin (optionally Eryc™, Erythrocin™), and optionally the ketolide antibiotic is telithromycin (Ketek™); metronidazole (optionally Flagyl™, Metro™); a polyketide antibiotic (optionally anthracimycin, geldanamycin, doxycycline (optionally Doryx™, Doxyhexal™, Doxylin™), erythromycin (optionally Eryc™, Erythrocin™); rifampicin (also known as rifampin) (optionally Rifadin™), a streptogramin, wherein optionally the streptogramin is pristinamycin (optionally Pyostacine™), quinupristin, dalfopristin, or both Quinupristin and dalfopristin (optionally Synercid™); a sulphonamide, wherein optionally the sulphonamide is hydrochlorothiazide (optionally Apo-hydro™), furosemide (optionally Lasix™) or sulfamethoxazole (optionally Gantanol™); tinidazole (optionally Fasigyn™, Simplotan™, Tindamax™); tigecycline (optionally Tygacil™); and trimethoprim (optionally Proloprim™, Monotrim™, Triprim™).

In alternative embodiments, to reduce liver dysfunction, uveitis, and leucopenia caused by administered antibiotics, particularly by antibiotics such as rifabutin, clarithromycin and clofazimine, the antibiotics are administered in a ramping-up in separate doses, and can be combined in a single tablet or capsule unit, but also can commence with a single drug, for example, clofazimine, then after 1 to 100 days add the second antibiotic, for example, rifabutin, also waiting 1 to 100 days before adding the third antibiotic, and so on if desired with additional antibiotics. In a blister pack or equivalent a placebo can be co-administered to allow simpler conceptualisation of dosing 1 by the patient (consistency of dosing), where at all times the same number of unit dosages, e.g., two, three, four our more pills, capsules or tablets, will be ingested. Such dosing may apply to double, triple, quad or other therapies.

Probiotics and Prebiotics

In alternative embodiments, additives, e.g., prebiotics and/or probiotics, are formulated with a therapeutic combination or composition as provided herein, are formulated for oral administration. In alternative embodiments, in methods as provided herein prebiotics and/or probiotics are administered (e.g., orally) before, during and/or after administration (treatment with) a therapeutic combination or composition as provided herein whether or not they are formulated with a therapeutic combination or composition as provided herein or formulated or packaged as entirely separate entities.

In alternative embodiments, additives that are included in a therapeutic combination or composition as provided herein, e.g., a tablet (e.g., an orally disintegrating sublingual tablet or buccal tablet or wafer) or geltab, liquid, gel, lozenge, dissolvable patch or wafer, lollie (e.g., lollypop, “pop” or sucker), candy, gum (e.g., a chewing gum), aerosol or spray preparation, or a composition used to practice a method as provided herein, includes one or more prebiotics and/or probiotics. In alternative embodiments, prebiotics and/or probiotics are administered are administered before, during (e.g., concurrent with) and/or after administration (treatment with) a therapeutic combination or composition as provided herein.

In alternative embodiments, the prebiotics can be inulin, apple pectin, lactulose, extracts of artichoke, chicory root, oats, barley, various legumes, garlic, kale, beans or flacks, herbs or N-acetyl glucosamine (GlcNAc) or equivalents thereof or combinations thereof.

In alternative embodiments, additives may include one, two, three or more flora components, including Firmicutes, Bacteroidetes, Proteobacteria, Actinobacteria, Spirochaetes, and Fusobacteria, Euryarchaeota, Chlamydia, Chloroflexi, SR1, Synergistetes, Tenericutes, and TM7. Probiotics, such as a Streptococcus, optionally a Streptococcus salivarius, optionally Streptococcus salivarius K12, Bacteroidetes, Firmicutes, Bacillus (e.g., Bacillus thurigiensis) or any combination thereof may be used. In alternative embodiments, cultured components are added back to the flora (e.g., of the administered probiotic) to fortify or expand on the amount or scope (variety) of specific genus or species administered, e.g., Bacteroidetes, Firmicutes, Bacillus or Bacillus thurigiensis. In alternative embodiments, probiotics are included as single cultured components; for some embodiments and species, multiply cultured components are avoided as they lose their implantation characteristics.

Preservatives, Cryoprotectants, Lyoprotectants

In alternative embodiments, to any therapeutic combination or composition as provided herein (e.g., the liquid preparation embodiment, including aerosols and sprays, the candies, lollies, gels, foods, drinks and the like as provided herein) may be added various preservatives, cryoprotectants and/or lyoprotectants, including e.g., various polysaccharides or sugars (such as sucrose, fructose, lactose, mannitol), glycerol, polyethylene glycol (PEG), trehalose, glycine, glucose, dextran and/or erythritol. In alternative embodiments, other cryoprotectants that can be used are ethylene glycol, 1,2-Propanediol, Methylcelliosolve, Dimethyl Formamide, or Dimethyl sulphoxide Methanol. In alternative embodiments the content of these cryoprotectants are between about 1% and about 50% but generally between about 5% and about 15% is adequate.

Because of the ability to freeze and/or freeze-dry, or spray dry, any therapeutic combination or composition as provided herein, in alternative embodiments there are different types of final products that can be manufactured. In alternative embodiments, a therapeutic combination or composition as provided herein is formulated as a liquid. In alternative embodiments, a product or formulation of the invention is frozen and kept at e.g. minus 80 degrees for usage later given a cryoprotectant is added.

Biofilm Disrupting Compounds

In alternative embodiments, one or more biofilm disrupting compounds, including compounds able to disrupt biofilms in the mouth or pharynx, e.g., a pharyngeal biofilm disrupting compound, is/are added into a therapeutic combination or composition as provided herein (e.g., a liquid preparation embodiment), or used to practice a method or use as provided herein. In alternative embodiments, in practicing the methods or uses as provided herein, biofilm disrupting compounds are administered before or during (co-administered), or co-formulated with (e.g., in a liquid, gel, food, tablet (e.g., an orally disintegrating sublingual tablet or buccal tablet or wafer or strip, or multilaminated tablet) or capsule, or aerosol or spray, or dissolvable patch), or separately formulated, as the administered composition or formulation of the invention. In alternative embodiments, disrupting biofilms are used to separate from pharyngeal or colonic mucosa an adherent polysaccharide/DNA—containing layer, the so-called “biofilm.

In alternative embodiments, other biofilm disrupting components or agents also can be used, e.g., enzymes such as a deoxyribonuclease (DNase), a N-acetylcysteine, an auranofin, alginate lyase, glycoside hydrolase dispersin B; Quorum-sensing inhibitors e.g., ribonucleic acid III inhibiting peptide, Salvadora persica extracts, Competence-stimulating peptide, Patulin and penicillic acid; peptides—cathelicidin-derived peptides, small lytic peptide, PTP-7 (a small lytic peptide, see e.g., Kharidia (2011) J. Microbiol. 49(4):663-8, Epub 2011 September 2), Nitric oxide, neo-emulsions; ozone, lytic bacteriophages, lactoferrin, xylitol hydrogel, synthetic iron chelators, cranberry components, curcumin, silver nanoparticles, Acetyl-11-keto-β-boswellic acid (AKBA), barley coffee components, probiotics, sinefungin, S-adenosylmethionine, S-adenosyl-homocysteine, Delisea furanones, N-sulfonyl homoserine lactones and/or macrolide antibiotics or any combination thereof.

In alternative embodiments, biofilm disrupting components or agents are administered before, during (for example, concurrent with) and/or after the administration of a composition or formulation comprising a therapeutic combination as provided herein, e.g., lozenges, dissolvable wafers, strip or patches, lollies (e.g., lollypops, “pops” or suckers), candies, gums (e.g., chewing gums), aerosols, powders and sprays. In alternative embodiments, biofilm disrupting agents are administered either before treatment and/or during and/or after treatment with a therapeutic combination or composition as provided herein. In alternative embodiments, biofilm disrupting agents are used singly or in combination.

In alternative embodiments, biofilm disrupting agents include particular enzymes and degrading substances including in N-acetylcysteine, deoxyribonuclease (DNase). Others would include Alginate, lyase and Glycoside hydrolase dispersin, Ribonucleic-acid-III inhibiting peptide (RIP), Salvadora persica extracts, Competence-stimulating peptide (CSP) Patulin (PAT) and penicillic acid (PA)/EDTA, Cathelicidin-derived peptides, Small lytic peptide, PTP-7, Nitric oxide, Chlorhexidine, Povidone-iodine (PI), Nanoemulsions, Lytic bacteriophages, Lactoferrin/xylitol hydrogel, Synthetic iron chelators, Cranberry components, Curcumin, Acetyl-11-keto-boswellic acid (AKBA), Barley coffee (BC) components, silver nanoparticles, azithromycin, clarithromycin, gentamicin, streptomycin and also Disodium EDTA. Ozone insufflations of the pharyngeal area, e.g., including one or more tonsils, or the pharynx, can also be used to disrupt the biofilm.

### Other Co-therapeutic Combinations

In alternative embodiment, therapeutic combinations as provided herein also can be combined with skin-active therapies such as a “halobetasol propionate and tazarotene treatment” (e.g., as provided by Valeant Pharmaceuticals International, Laval, Quebec, Canada). In this way a systemic anti-Streptococcal treatment combines with a skin-active therapy accelerating the healing so increasing efficacy above that of either therapy. Similarly the current invention targeting the underlying tonsilar infections can be combined with a 11-17 inhibitor, brodalumab, working effectively at the skin level (SILIQ, Valeant), and brodalumab's most common adverse effects of nasopharyngitis are largely abolished, and the synergistic effect brings closer to 90% the response of the psoriasis, with increased number reaching full clearance or 100 PASI scores.

In alternative embodiments, therapeutic combinations as provided herein are combined with immune modulators, such as e.g., alefacept (optionally Amevive™), efalizumab (optionally Raptiva™), etanercept (optionally Enbrel™), ixekinumab (optionally Talz™), golimumab (optionally Simponi™), guselkumab (optionally Tremfya™), cetrolizumab-pegol (optionally Cimzia™), tildrakizumab (optionally Ilumya™), abatacept (optionally Orencia™), an anti-TNF infusion or product (e.g., adalimumab (optionally Humira™, Exemptia™) or infliximab (optionally Remicade™, Remsima™, Inflectra™)), anti-IL 12/23 (optionally ustekinumab, or Stelara™), anti-IL 23, anti-IL 17F, or anti-IL 17A (optionally secukinumab, or Cosentyx™) products, prednisone (optionally Deltasone™, Liquid Pred™, Orasone™, Adasone™, Deltacortisone™, Prednisonum™), cyclosporine or ciclosporine (optionally Neoral™, Sandimmune™), mercaptopurine or 6-MP (6-mercaptopurine) (optionally Purinethol™), methotrexate or amethopterin (optionally Trexall™, Rheumatrex™), tacrolimus (fujimycin) (optionally Prograf™ Advagraf™, Protopic™), ascomycin or immunomycin, rapamycin or sirolimus (optionally Rapamune™), sulfasalazine (optionally Azulfidine™, Salazopyrin™, Sulazine™), a steroid (e.g., a corticosteroid, or methylprednisolone (optionally Depo-Medrol™, Solu-Medrol™), azathioprine (optionally Azasan™, Imuran™) or a combination thereof. In alternative embodiments, a main advantage could be more rapid response, higher % response, longer response, reduced fall in efficacy over time, and/or a reduction in or early reversal of adverse effects.

### Unit Dosage Forms and Formulations, Foods, and Delivery Vehicles

In alternative embodiments, a therapeutic combination or composition as provided herein is manufactured, labelled or formulated as a liquid, a suspension, a powder, a spray, a gel, a geltab, a semisolid, a tablet, or sachet, a capsule, a lozenge, an orally dissolvable wafer, strip or patch, a chewable or suckable unit dosage form, or any pharmaceutically acceptable formulation or preparation.

In alternative embodiments, a therapeutic combination or composition as provided herein is incorporated into a food or a drink (e.g., a yogurt, gelato, ice, ice cream, smoothie), a candy, sweet or lolly (e.g., lollypop, pop or sucker), or a feed, a nutritional or a food or feed supplement (e.g., liquid, semisolid or solid), and the like.

In alternative embodiments, a therapeutic combination or composition as provided herein (e.g., a liquid preparation embodiment) can be further processed by, e.g., spray-drying or equivalent, e.g., spray-drying in an inert gas or freeze-drying under similar conditions, thus ending up with a powdered product.

In alternative embodiments, all the ingredients of a therapeutic combination or composition as provided herein are formulated in one unit dosage, e.g., in one tablet, capsule, geltab, liquid, suspension, powder, spray, food, drink, candy, lolly, gum and the like. In alternative embodiments, in addition to one or all of the ingredients of a therapeutic combination or composition as provided herein, also provided in a single unit dosage (e.g., single tablet, capsule, geltab, or single serving of a food or a liquid, or a single dosage of a spray, aerosol or powder e.g., from an inhaler or nebulizer), e.g., a single unit dosage also comprises: an additional compound or agent, including an active agent such as a prebiotic, probiotic or biofilm disrupting agent, and/or an non-(pharmaceutically) active agent such as a preservative, filler, colouring or a flavouring.

In alternative embodiments, a therapeutic combination or composition as provided herein is formulated to maximally expose the pharyngeal or the tonsillar area, optionally including the entire Waldeyer ring, to the therapeutic combinations and compositions as provided herein, thereby permitting absorption of active agents, e.g., antibiotics, into pharyngeal or the tonsillar area tissues to either supplement or replace systemic delivery of the same or different active agents, e.g., antibiotics. In alternative embodiments, one or more active agents, e.g., antibiotics, of a therapeutic combination or composition as provided herein is/are administered for direct absorption into pharyngeal or the tonsillar area tissue, while another one or more of the therapeutic combination or composition as provided herein are administered systemically.

For example, a therapeutic combination or composition as provided herein can be manufactured, labelled or formulated as an orally disintegrating tablet as described e.g., in U.S. Pat. App. Publication No. 20100297031. A therapeutic combination or composition as provided herein can be a polyol/thickened oil suspension as described in U.S. Pat. Nos. 6,979,674; 6,245,740. A therapeutic combination or composition as provided herein can be encapsulated, e.g., encapsulated in a glassy matrix as described e.g., in U.S. Pat. App. Publication No. 20100289164; and U.S. Pat. No. 7,799,341. A therapeutic combination or composition as provided herein can be manufactured, labeled or formulated as an excipient particle, e.g., comprising a cellulosic material such as microcrystalline cellulose in intimate association with silicon dioxide, a disintegrant and a polyol, sugar or a polyol/sugar blend as described e.g., in U.S. Pat. App. Publication No. 20100285164. A therapeutic combination or composition as provided herein n can be manufactured, labeled or formulated as an orally disintegrating tablet as described e.g., in U.S. Pat. App. Publication No. 20100278930. A therapeutic combination or composition as provided herein can be manufactured, labeled or formulated as a spherical particle, as described e.g., in U.S. Pat. App. Publication No. 20100247665, e.g., comprising a crystalline cellulose and/or powdered cellulose. A therapeutic combination or composition as provided herein can be manufactured, labeled or formulated as a rapidly disintegrating solid preparation useful e.g. as an orally-disintegrating solid preparation, as described e.g., in U.S. Pat. App. Publication No. 20100233278. A therapeutic combination or composition as provided herein can be manufactured, labeled or formulated as a solid preparation for oral application comprising a gum tragacanth and a polyphosphoric acid or salt thereof, as described e.g., in U.S. Pat. App. Publication No. 20100226866, and may be presented as an attractively-tasting chewing gum.

A therapeutic combination or composition as provided herein can be manufactured, labeled or formulated using a water soluble polyhydroxy compound, hydroxy carboxylic acid and/or polyhydroxy carboxylic acid, as described e.g., in U.S. Pat. App. Publication No. 20100222311. A composition of the invention can be manufactured, labeled or formulated as a lozenge, or a chewable and suckable tablet or other unit dosage form, as described e.g., in U.S. Pat. App. Publication No. 20100184785.

A therapeutic combination or composition as provided herein can be manufactured, labeled or formulated in the form of an agglomerate, as described e.g., in U.S. Pat. App. Publication No. 20100178349. A therapeutic combination or composition as provided herein can be manufactured, labeled or formulated in the form of a gel or paste, as described e.g., in U.S. Pat. App. Publication No. 20060275223. A therapeutic combination or composition as provided herein can be manufactured, labeled or formulated in the form of a soft capsule, as described e.g., in U.S. Pat. No. 7,846,475, or U.S. Pat. No. 7,763,276.

The polyols used in a therapeutic combination or composition as provided herein can be micronized polyols, e.g., micronized polyols, e.g., as described e.g., in U.S. Pat. App. Publication No. 20100255307, e.g., having a particle size distribution (d₅₀) of from to 60 μm, and a flowability below or equal to 5 s/100 g, or below 5 s/100 g.

Gradual or Delayed Release Formulations

In alternative embodiments, provided are therapeutic combinations or compositions as provided herein formulated for delayed, chronodosed or gradual oral or enteric release comprising at least one active agent (e.g., a formulation or pharmaceutical preparation of the invention) formulated with a delayed release composition or formulation, coating or encapsulation. This would permit prolonged antibiotic release with reduced troughs and peaks. In alternative embodiments, formulations or pharmaceutical preparations as provided herein are designed or formulated for delivery of active ingredient, e.g., into the distal small bowel.

In alternative embodiments, a formulation or pharmaceutical preparation as provided herein is a liquid formulation, an oral microbiota-comprising formulation, which can be a frozen or a freeze-dried preparation. In alternative embodiments, e.g., for the encapsulated format, all are in powdered form.

In alternative embodiments, therapeutic combinations or compositions as provided herein are formulated for delayed or gradual oral or enteric release using cellulose acetate (CA) and polyethylene glycol (PEG), e.g., as described by Defang et al. (2005) Drug Develop. & Indust. Pharm. 31:677-685, who used CA and PEG with sodium carbonate in a wet granulation production process.

In alternative embodiments, therapeutic combinations or compositions as provided herein are formulated for delayed or gradual oral or enteric release using a hydroxypropylmethylcellulose (HPMC), a microcrystalline cellulose (MCC) and magnesium stearate, as described e.g., in Huang et al. (2004) European J. of Pharm. & Biopharm. 58: 607-614).

In alternative embodiments, therapeutic combinations or compositions as provided herein are formulated for delayed or gradual oral or enteric release using e.g., a poly(meth)acrylate, e.g. a methacrylic acid copolymer B, a methyl methacrylate and/or a methacrylic acid ester, a polyvinylpyrrolidone (PVP) or a PVP-K90 and a EUDRAGIT® RL PO™, as described e.g., in Kuksal et al. (2006) AAPS Pharm. 7(1), article 1, E1 to E9.

Feeds, Drinks, Candies, Nutritional or a Food or Feed Supplements

In alternative embodiments, therapeutic combinations or compositions as provided herein, and/or prebiotics and/or oral probiotics as provided herein, are incorporated into a food, a feed, a candy (e.g., a lollypop or a lozenge) a drink, a nutritional or a food or feed supplement (e.g., liquid, semisolid or solid), and the like, as described e.g., in U.S. Pat. App. Publication No. 20100178413. In one embodiment, therapeutic combinations or compositions as provided herein are incorporated into (manufactured as) a beverage as described e.g., in U.S. Pat. No. 7,815,956. For example, a therapeutic combinations or compositions as provided herein are incorporated into a yogurt, an ice cream, a milk or milkshake, a “frosty”, “snow-cone”, or other ice-based mix, and the like.

In alternative embodiments, therapeutic combinations or compositions as provided herein, and/or prebiotics and/or oral probiotics as provided herein, are contained in, are formulated as or comprise a freeze-dried powder form added to a food, e.g., a yogurt, an ice cream, a gelato, a juice, a milk or milkshake, a “frosty”, “snow-cone”, or other ice-based mix, and the like. In embodiment, it can be kept in a lid-storage (e.g., of a yogurt or ice cream) such that when it is twisted the powder falls into the product or formulation (e.g., yoghurt or ice cream, or liquid or drink) and then it can be stirred so as not to have the powder ferment ‘standing on the shelf’. Various flavourings can be added.

In alternative embodiments, the probiotics comprise a Firmicutes, Bacteroidetes, Proteobacteria, Actinobacteria, Spirochaetes, and Fusobacteria, Euryarchaeota, Chlamydia, Chloroflexi, SR1, Synergistetes, Tenericutes, and TM7. Probiotics, such as a Streptococcus, optionally a Streptococcus salivarius, optionally Streptococcus salivarius K12, Bacteroidetes, Firmicutes, Bacillus (e.g., Bacillus thurigiensis) or any combination thereof may be used.

### Methods of Use and Applications of Compositions of the Invention

In alternative embodiments, therapeutic combinations or compositions as provided herein, and/or a method or a use are provided herein, are used to treat, ameliorate, prevent or reverse: a skin disease, infection, reaction or condition; a neurological disease or syndrome, or a genetically-predisposed or a chronic neurological disorder, where the microbial or bacterial flora (normal or abnormal, e.g., as in infected tissue) of the affected tissue is at least one causative or symptom-producing factor.

In alternative embodiments, therapeutic combinations or compositions as provided herein, and/or a method or a use are provided herein, are used to treat, ameliorate, prevent or reverse a bacterially-induced disease, condition, infection or reaction in the individual in need thereof, e.g., an autoimmune disease, condition, infection or reaction, wherein optionally the autoimmune disease or condition is: a skin or skin-related autoimmune disease or condition, and optionally the skin or skin-related autoimmune disease or condition is a psoriasis, optionally a plaque-, guttate-, inverse-, pustular- or erythrodermic-type psoriasis; or a Pustulosis Palmaris et Plantaris (PPP) or a Palmoplantar pustulosis; a Pediatric Autoimmune Neuropsychiatric Disorder (e.g., a Pediatric Autoimmune Neuropsychiatric Disorder Associated with a Streptococcal Infection (PANDAS)); rheumatic fever or rheumatic heart disease; reactive arthritis; or, acute glomerulonephritis.

In alternative embodiments, therapeutic combinations or compositions as provided herein, and/or a method or a use are provided herein, are used to treat or ameliorate PANDAS in patients, e.g., patients having a sudden acute and debilitating onset of intense anxiety and mood lability accompanied by Obsessive Compulsive-like issues and/or Tics in association with a Streptococcal-A (GABHS) infection that has occurred immediately prior to the symptoms. In some instances, the onset will be 4 to 6 months after a Streptococcal infection because the initial course of antibiotics did not fully eradicate the bacteria.

The therapeutic combinations may be used in full dosage, then in a reduced maintenance dosage, which can be followed by use of lozenges or the like to replace partially eradicated pathogenic Streptococcal infections in the lymphoid tissue of the tonsillar tissue with a non-pathogenic strain, e.g., of Streptococcu salivarius, or other normal oral macro-biota. As a further alternative, after antibiotic and combination antibiotic treatment, tonsillectomy or tonsillectomy and adenoid removal may be done, to prevent relapse of the condition, infection or reaction.

### Packaging

The invention provides therapeutic combinations or compositions, including preparations, formulations and/or kits, comprising combinations of ingredients, as described herein. In alternative embodiments, these combinations can be mixed and administered together, or alternatively, they can be an individual member of a packaged combination of ingredients, e.g., as manufactured in a separate package, kit or container; or, where all or a subset of the combinations of ingredients are manufactured in a separate package or container. In alternative aspects, the package, kit or container comprises a blister package, a clamshell, a tray, a shrink wrap and the like.

In one aspect, the package, kit or container comprises a “blister package” (also called a blister pack, or bubble pack). In one aspect, the blister package is made up of two separate elements: a transparent plastic cavity shaped to the product and its blister board backing. These two elements are then joined together with a heat sealing process which allows the product to be hung or displayed. Exemplary types of “blister packages” include: Face seal blister packages, gang run blister packages, mock blister packages, interactive blister packages, slide blister packages.

Blister packs, clamshells or trays are forms of packaging used for goods; thus, the invention provides for blister packs, clamshells or trays comprising a composition (e.g., a (the multi-ingredient combination of drugs of the invention) combination of active ingredients) of the invention. Blister packs, clamshells or trays can be designed to be non-reclosable, so consumers can tell if a package has already opened. They are used to package for sale goods where product tampering is a consideration, such as the pharmaceuticals of the invention. In one aspect, a blister pack as provided herein comprises a moulded PVC base, with raised areas (the “blisters”) to contain the tablets, pills, etc. comprising the combinations of the invention, covered by a foil laminate. Tablets, pills, etc. are removed from the pack either by peeling the foil back or by pushing the blister to force the tablet to break the foil. In one aspect, a specialized form of a blister pack is a strip pack. In one aspect, in the United Kingdom, blister packs adhere to British Standard 8404.

### Devices and Products of Manufacture

In alternative embodiments, therapeutic combinations as provided herein are delivered by and/ or contained in: a device, e.g., a medical device; an implant, e.g., a breast implant; a prosthesis; a stent; a catheter; a spray; a nebulizer; an atomizing or an aerosol device; or an inhaler or neck implant. In alternative embodiments, the spray, aerosol device or inhaler provide oral delivery of liquids or powders comprising therapeutic combinations as provided herein. In alternative embodiments, nebulizers, inhalers or atomizing devices used to orally delivery liquids or powders comprising therapeutic combinations as provided herein can be as described or used in U.S. Pat. Nos. 9,566,398; 9,533,122; 9,415,008; 9,308,334; 7,571,722.

In alternative embodiments, therapeutic combinations as provided herein are delivered by and/ or contained in: an orally dissolvable strip, wafer or patch, which optionally can comprise water-soluble or water-miscible polymers, including e.g., a cellulose, cellulose derivatives, synthetic or natural gums, such as xanthan gum, tragacanth gum, guar gum, acacia gum, arable gum, Locust bean gum, methacrylic acid polymers, methacrylic acid copolymers, acrylic acid polymers, acrylic acid copolymers, polyacrylamides, polyalkylene oxides, polyalkylene glycols, carrageanan, pullunan, locust bean gums, bean starches, polyvinyl pyrrolidone, polyvinyl alcohol, alginic acid, salts of alginic acid, carboxyvinyl polymers, pectin, pectin derivatives, xanthan gum, xanthan gum derivatives, pea starch, starch starch derivatives, carrageanan, alginic acid, salts of alginic acid and mixtures thereof. In alternative embodiments, gums used to make an orally dissolving strip, wafer or patch comprises xanthan gum, tragacanth gum, guar gum, acacia gum, arable gum, locust bean gum, and mixtures thereof In alternative embodiments, cellulose derivatives include methyl cellulose, ethyl cellulose, hydroxyethyl cellulose, hydroxypropyl methyl cellulose, carboxymethyl cellulose, sodium carboxymethyl cellulose, and mixtures thereof. In alternative embodiments, orally dissolvable formulations as provided herein comprise a matrix of polyvinyl pyrrolidone and/or polymeric alginate.

In one embodiment, an orally dissolvable formulation as provided herein includes a hydrophobic pressure-sensitive adhesive or bioadhesive to provide a desired control of tack, adhesive and water sorption properties required for application to a mucosal tissue; see e.g., U.S. Pat. Nos. 5,166,233: 6,552,024: 7,906,140: 6,803,420; 7,984,714; 7,276,246; 5,578,315; 7,470,397; 7,138,135 and 7,441,559; and publications U.S. Pat. Publication No. 20110033542.

The invention will be further described with reference to the following examples; however, it is to be understood that the invention is not limited to such examples.

### EXAMPLES

### Example 1: Exemplary Treatment of Psoriasis in Adults with OCD Using Metronidazole

A twenty-three year old patient with Obsessive-Compulsive Personality Disorder (OCD) commenced treatment in April 2016 with two non-absorbable antibiotics (vancomycin and rifaximin), together with the absorbable Metronidazole. The aim was to suppress his gut bacteria to reduce the OCD. His scalp was also involved with extensive psoriasis and dandruff like scales.

Progressively, his behavioural changes were significantly suppressed, his diarrhoea and cramping and other accompanying symptom also improved markedly. By November of 2016, it was observed that the Metronidazole resulted in marked suppression of the psoriasis of the scalp. By February of 2017, there was very little psoriasis left on the scalp, and the OCD was improved, for example, patient was less aggressive and more settled. This is an example of a monotherapy using Metronidazole to treat (e.g., suppress) psoriasis.

### Example 2: Exemplary Treatment of Psoriasis with Vancomycin and Ceftriaxone

A fifty-seven year old patient with quite extensive psoriasis on his back, buttocks, arms, and legs developed acute meningitis with nuchal rigidity, high fevers, profound light sensitivity, nausea, and vomiting. He was admitted to hospital in July 2011 and baseline blood tests were taken but unfortunately he was given antibiotics prior to reaching the teaching hospital, and even the lumbar puncture fluid did not grow a pathogen.

Nevertheless, intravenous Vancomycin was commenced together with intravenous Ceftriaxone. Both of these antibiotics were given over a period of four weeks while his fever settled and his pains and aches progressively improved. After discharge from the hospital he noticed that the extensive psoriasis was completely gone. Over the next two and a half years there was no recurrence of the psoriasis.

### Example 3: Exemplary Treatment of Psoriasis with Rifabutin, Clofazimine, and Clarithromycin

A patient with Crohn's disease and extensive psoriasis of the elbows, hands, knees, ankles, back and buttocks—was commenced on treatment for mycobacterium avium paratuberculosis in January 2011 starting with oral Rifabutin, Clofazimine, and Clarithromycin. He was told there was a good chance his psoriasis may well improve, especially since Clofazimine was known to inhibit this condition and Rifabutin possessed anti-inflammatory characteristics. The medication doses were progressively ramped up and by week 6 reached 600 mg/d Rifabutin, 150 mg/d Clofazimine, and 1 g/d of Clarithromycin. It was noticed that not only his inflammatory bowel disease slowly improved but his psoriasis, which was quite extensive, progressively disappeared, with new skin forming and outer layers peeling off. He was on treatment for three years. The psoriasis completely disappeared, and only 2 years after ceasing the antibiotics did some re-emergence of the skin lesions begin. Blis lozenges containing Strep salivarius were added as a daily therapy, and the relapse stopped completely and the severe changes have not reoccurred.

### Example 4: Exemplary Treatment of Psoriasis with Clofazimine, Rifampicin, Tinidazole, Enbrel™

A 38 year (y) old male suffered with extensive psoriasis becoming dependent on steroids and gaining much weight. In spite of the steroids, he had clear extensive plaque psoriasis visible on areas of his face, elbows, arms, finger joints, knees, buttocks and ankles. Methotrexate caused unacceptable adverse effects and he was commenced on Humira™—which did not seem to help adequately, then changed to etanercept (Enbrel™). He noted a response with reduction of the surface area of his body covered by psoriasis plaque. But then the response stalled in spite of many months of injections. It was at this time that antibiotics were added to the etanercept (Enbrel™). He commenced on clofazimine and rifampicin and his skin lesions cleared almost completely. To try and obtain a 100% response, he added tinidazole, just 250 mg bid, and even his scalp completely cleared up. He now was no detectable psoriasis lesions anywhere on his body and is maintaining etanercept (Enbrel™) and the 3 antibiotics (clofazimine, rifampicin, tinidazole) for over 13 months.

A number of embodiments of the invention have been described. Nevertheless, it will be understood that various modifications may be made without departing from the spirit and scope of the invention. Accordingly, other embodiments are within the scope of the following claims.

What is claimed is:

1. A therapeutic combination, or a composition, for treating, ameliorating or preventing a bacterially-induced condition, disease, infection or reaction in an individual in need thereof, or treating or ameliorating a bacterially-affected or a bacterially-infected tissue,
wherein optionally the bacterially-induced condition, disease, infection or reaction is a Streptococcal-Induced condition, or the bacterially-affected or a bacterially-infected tissue comprises a Streptococcal-affected or a Streptococcal-infected tissue,
wherein optionally the individual active agents (e.g., each individual antibiotic) of the therapeutic combination, or the composition are administered together (simultaneously), in a step-wise fashion, or lagging one by one to separate administration of each active agent,
and optionally the bacterially-induced condition, disease, infection or reaction is located in a tonsil, and optionally the tonsil is a palatine tonsil, an adenoid, and/or a tonsillar tissue at the base of the tongue or next to an eustachian tube,
wherein the therapeutic combination, or the composition, comprises:
(a)
(1) clofazimine (optionally Lamprene™), an ansamycin, and amoxicillin (optionally Augmentin™, Amoxil™, Tyclav™, Synulox™, Dispermox™, Trimox™, Moxatag™)
(2) clofazimine (optionally Lamprene™), an ansamycin (optionally rifabutin), amoxicillin (optionally Augmentin™, Amoxil™, Tyclav™, Synulox™, Dispermox™, Trimox™, Moxatag™) and clavulanic acid,
(3) clofazimine (optionally Lamprene™) and an ansamycin,
(4) clofazimine (optionally Lamprene™), an ansamycin, and a combination of amoxicillin and clavulanic acid (Clavulin™),
wherein optionally the ansamycin is rifabutin (optionally Mycobutin™), rifampicin (also known as rifampin) (optionally Rifadin™), rifalazil (also known as KRM-1648 and ANTI-1648) or a combination thereof,
and optionally:
(i) the clofazimine is administered or formulated for administration at a dose in the range of between about 10 to 800 mg/d (day), or between about 1.0 to 1000 mg/d (day)
(ii) the rifabutin is administered or formulated for administration at a dose in the range of between about 10 to 900 mg/d (day), or between about 5 to 1200 mg/d (day),
(iii) the rifabutin or rifampicin is administered or formulated for administration at a dose in the range of between about 10 to 900 mg/d (day), or between about 5 to 1200 mg/d (day),
(iv) the amoxicillin and/or clavulanic acid is/are individually administered, or combined with another active agent, or formulated for administration at a dose in the range of between about 10 to 2,000 mg/d (day), between about 5 to 4,000 mg/d (day), and optionally the another active agent is an amoxicillin (optionally Augmentin™, Amoxil™, Tyclav™, Synulox™, Dispermox™, Trimox™, Moxatag™), or
(v) any combination of (i) to (iv), or all of (i) to (iv);
(b)
(1) rifabutin (optionally Mycobutin™), a macrolide antibiotic, and clofazimine (optionally Lamprene™)
(2) rifabutin (optionally Mycobutin™) and a macrolide antibiotic,
(3) a macrolide antibiotic and clofazimine (optionally Lamprene™),
(4) rifabutin (optionally Mycobutin™) and clofazimine (optionally Lamprene™); or rifabutin (optionally Mycobutin™), clofazimine (optionally Lamprene™) and a macrolide antibiotic; or rifabutin (optionally Mycobutin™) and a macrolide antibiotic,
wherein optionally the macrolide antibiotic is clarithromycin (optionally Biaxin™), azithromycin (optionally Zithromax™, Azithrocin™), roxithromycin, erythromycin (optionally Eryc™, Erythrocin™) or a combination thereof,
wherein optionally the rifabutin is administered or formulated for administration at a dose in the range of between about 100 to 200 mg/dose, or for administration at a dosage of about between about 100 to 200 mg/d (day), or at a dosage of about 150 mg/d,
and optionally the clofazimine is administered or formulated for administration at a dose in the range of between about 25 to 150 mg/dose, or is administered or formulated for administration at a dose in the range of between about 25 to 150 mg/d,
and optionally the macrolide antibiotic (optionally clarithromycin) is administered or formulated for administration at a dose in the range of between about 50 to 300 mg/dose, or is administered or formulated for administration at a dose in the range of between about 50 to 300 mg/d, or at about 250 mg/d;
(c)
(1) clofazimine (optionally Lamprene™) and ciprofloxacin (optionally Ciloxan™, Cipro™, Neofloxin™),
(2) ciprofloxacin (optionally Ciloxan™, Cipro™, Neofloxin™) and ansamycin, or
(3) clofazimine (optionally Lamprene™), ciprofloxacin (optionally Ciloxan™, Cipro™, Neofloxin™) and an ansamycin,
wherein optionally the ansamycin is rifabutin (optionally Mycobutin™), rifampicin (also known as rifampin) (optionally Rifadin™), rifalazil (also known as KRM-1648 and ANTI-1648) or a combination thereof,
(d) clofazimine (optionally Lamprene™), clindamycin (optionally Cleocin™, Dalacin™, Clinacin™) and clarithromycin (optionally Biaxin™);
(e) clofazimine (optionally Lamprene™), azithromycin (optionally Zithromax™, Azithrocin™) and cephalexin (also called cephalexin) (optionally Keflex™, Cepol™, Ceporex™);
(f) clofazimine (optionally Lamprene™), rifampicin (also known as rifampin) (optionally Rifadin™), and metronidazole (optionally Flagyl™, Metro™) or Tinidazole;
(g) rifampicin (also known as rifampin) (optionally Rifadin™), clofazimine (optionally Lamprene™), clarithromycin (optionally Biaxin™) and tinidazole (optionally Fasigyn™, Simplotan™, Tindamax™);
(h) amoxicillin (optionally Augmentin™, Amoxil™, Tyclav™, Synulox™, Dispermox™, Trimox™, Moxatag™), metronidazole (optionally Flagyl™, Metro™) and azithromycin (optionally Zithromax™, Azithrocin™);
(i) ciprofloxacin (optionally Ciloxan™, Cipro™, Neofloxin™), clofazimine (optionally Lamprene™) and amoxicillin (optionally Augmentin™, Amoxil™, Tyclav™, Synulox™, Dispermox™, Trimox™, Moxatag™),
wherein optionally the ciprofloxacin can be substituted with any quinolone or fluoroquinolone, wherein optionally the quinolone or the fluoroquinolone is moxifloxacin (optionally Avelox™, Avalox™, Avelon™, Vigamox™, Moxeza™), levofloxacin (optionally Levaquin™, Tavanic™, Iquix™), norfloxacin (optionally Noroxin™), ofloxacin (optionally Floxin™, Ocuflox™) or gemifloxacin (optionaly Factive™);
(j) metronidazole (optionally Flagyl™, Metro™, optionally an absorbable metronidazole) alone; or metronidazole (optionally Flagyl™, Metro™) and vancomycin (optionally Vancocin™, or a formulation as described in WO 2014085526 A1, optionally intravenously (IV) administered vancomycin (formulated for IV administration); or metronidazole (optionally Flagyl™, Metro™) and rifaximin (optionally Xifaxan™, Xifaxanta™, Normix™); or metronidazole (optionally Flagyl™, Metro™, optionally an absorbable metronidazole), vancomycin (optionally Vancocin™, or a formulation as described in WO 2014085526 A1, optionally intravenously (IV) administered vancomycin (formulated for IV administration)) and rifaximin (optionally Xifaxan™, Xifaxanta™, Normix™);
(k) a bactericidal lipoglycopeptide and a cephalosporin (optionally vancomycin (optionally Vancocin™, or a formulation as described in WO 2014085526 A1, optionally intravenously (IV) administered vancomycin (formulated for IV administration) and ceftriaxone (optionally Rocephin™, Epicephin™)),
wherein optionally the cephalosporin is ceftaroline fosamil (optionally Teflaro™, Zinforo™), cephacetrile, cefadroxyl (optionally Duricef™), cephalexin (also called cephalexin) (optionally Keflex™, Cepol™, Ceporex™); cephaloglycin, cephalonium, cephaloridine, cephalothin (optionally Keflin™) cephapirin (optionally Cefadryl™), cefatrizine, cefazaflur, cefazedone, cefazolin (or cephazolin; optionally Ancef™, Kefzol™), cefradine (or cephradine; optionally Velosef™), cefaclor (optionally Ceclor™, Distaclor™, Keflor™, Raniclor™), cefonicid (optionally Monocid™), cefprozil (or cefproxil, optionally Cefzil™), cefuroxime (optionally Zefu™, Zinnat™, Zinacef™, Ceftin™, Biofuroksym™, Xorimax™), loracarbef (optionally Lorabid™), cefmetazole (optionally Zefazone™), cefotetan (optionally Cefotan™), cefoxitin (optionally Mefoxin™), cefotiam (optionally Pansporin™), cefdinir (optionally Sefdin™, Zinir™, Omnicef™, Kefnir™), cefixime (optionally Fixx™, Zifi™, Suprax™), cefotaxime (optionally Claforan™), cefovecin (optionally Convenia™), cefpodoxime (optionally Vantin™, PECEF™, Simplicef™), cefteram, ceftamere (optionally Enshort™), ceftibuten (optionally Cedax™), ceftiofur (optionally Naxcel™, Excenel™), ceftiolene, ceftizoxime (optionally Cefizox™), ceftriaxone (optionally Rocephin™, Epicephin™), cefoperazone (optionally Cefobid™), ceftazidime (optionally Meezat™, Fortum™, Fortaz™), cefepime (optionagarglly Maxipime™, Voco™), cefpirome (optionally Cefrom™), or flomoxef,
wherein optionally the bactericidal lipoglycopeptide is telavancin (optionally Vibativ™), and optionally the lipopeptide antibiotic is daptomycin (Cubicin™), and optionally the glycopeptide is bleomycin (Blenoxane™), teicoplanin (Targocid™), or a vancomycin (optionally Vancocin™, or a formulation as described in WO 2014085526 A1, optionally intravenously (IV) administered vancomycin (formulated for IV administration); and/or
(l) a selection or combination of at least one, two, three, four, five, six or seven or more of any antibiotic or antibiotics selected from:
amikacin (optionally Amikin™);
an aminoglycoside,
wherein optionally the aminoglycoside is amikacin (optionally Amikin), tobramycin (optionally Tobrex™), dibekacin, kanamycin, gentamycin (optionally Cidomycin™, Septopal™, Genticyn™, Garamycin™), sisomicin (also known as ensamycin) (optionally bactoCeaze™), neomycin (optionally Neo-rx™), netilmicin, paromomycin (optionally Catenulin™, Aminosidine™) or streptomycin;
amoxicillin (optionally Augmentin™, Amoxil™, Tyclav™, Synulox™, Dispermox™, Trimox™, Moxatag™);
an ansamycin,
wherein optionally the ansamycin is rifabutin (optionally Mycobutin™), rifampicin (also known as rifampin) (optionally Rifadin™), rifalazil (also known as KRM-1648 and ANTI-1648) or a combination thereof;
azithromycin (optionally Zithromax™, Azithrocin™);
a beta-lactam antibiotic,
wherein optionally the beta-lactam antibiotic is a penicillin a carbapenem, a cephalosporin, or a monobactam,
and optionally the penicillin is benzylpenicillin (also known as penicillin G) (optionally Pfizerpen™), benzathine benzylpenicillin (also known as benzathine penicillin G), phenoxymethylpenicillin (also known as penicillin V) (optionally Veetids™), or procaine benzylpenicillin (also known as penicillin G procaine) (optionally Bicillin C-R™),
and optionally the carbapenem is imipenem (optionally Primaxin™), meropenem (optionally Merrem™), ertapenem (optionally Invanz™), doripenem (optionally Finibax™, Doribax™), panipenem (also called betamipron), biapenem, razupenem (optionally PTZ-601™), tebipenem (optionaly Orapenem™),
and optionally the monobactam is aztreonam (optionally Azactam™, Cayston™), tigemonam, nocardicin A, tabtoxin,
and optionally the cephalosporin is ceftaroline fosamil (optionally Teflaro™, Zinforo™), cephacetrile, cefadroxyl (optionally Duricef™), cephalexin (also called cephalexin) (optionally Keflex™, Cepo1™, Ceporex™); cephaloglycin, cephalonium, cephaloridine, cephalothin (optionally Keflin™), cephapirin (optionally Cefadryl™), cefatrizine, cefazaflur, cefazedone, cefazolin (or cephazolin; optionally Ancef™, Kefzol™), cefradine (or cephradine; optionally Velosef™), cefaclor (optionally Ceclor™, Distaclor™, Keflor™, Raniclor™), cefonicid (optionally Monocid™), cefprozil (or cefproxil, optionally Cefzil™), cefuroxime (optionally Zefu™, Zinnat™, Zinacef™, Ceftin™, Biofuroksym™, Xorimax™), loracarbef (optionally Lorabid™), cefmetazole (optionally Zefazone™), cefotetan (optionally Cefotan™), cefoxitin (optionally Mefoxin™), cefotiam (optionally Pansporin™), cefdinir (optionally Sefdin™, Zinir™, Omnicef™, Kefnir™), cefixime (optionally Fixx™, Zifi™, Suprax™), cefotaxime (optionally Claforan™), cefovecin (optionally Convenia™), cefpodoxime (optionally Vantin™, PECEF™, Simplicef™), cefteram, ceftamere (optionally Enshort™), ceftibuten (optionally Cedax™), ceftiofur (optionally Naxcel™, Excenel™), ceftiolene, ceftizoxime (optionally Cefizox™), ceftriaxone (optionally Rocephin™, Epicephin™), cefoperazone (optionally Cefobid™), ceftazidime (optionally Meezat™, Fortum™, Fortaz™), cefepime (optionagarglly Maxipime™, Voco™), cefpirome (optionally Cefrom™), or flomoxef;
clarithromycin (optionally Biaxin™);
clavulanic acid;
clofazimine (optionally Lamprene™);
ethambutol (optionally Myambutol™, Etibi™, Servambutol™);
fosomycin (optionally Monurol™, Monuril™);
a lincosamide,
wherein optionally the lincosamide is lincomycin, pirlimycin, or clindamycin (optionally Cleocin™, Dalacin™, Clinacin™);
a bactericidal lipoglycopeptide, a glycopeptide, or a lipopeptide antibiotic,
wherein optionally the bactericidal lipoglycopeptide is telavancin (optionally Vibativ™), and optionally the lipopeptide antibiotic is daptomycin (Cubicin™), and optionally the glycopeptide is bleomycin (Blenoxane™), teicoplanin (Targocid™), or a vancomycin (optionally Vancocin™, or a formulation as described in WO 2014085526 A1, optionally intravenously (IV) administered vancomycin (formulated for IV administration);
a nystatin (optionally Mycostatin™, Nystop™),
a oxazolidinone,
wherein optionally the oxazolidinone is linezolid (optionally Zyvox™, Zyvoxid™), posizolid, tedizolid (optionally Sivextro™), radezolid (optionally RX-1741™) or cycloserine (optionally Seromycin™),
a quinolone or a fluoroquinolone,
wherein optionally the quinolone or the fluoroquinolone is moxifloxacin (optionally Avelox™, Avalox™, Avelon™, Vigamox™, Moxeza™), ciprofloxacin (optionally Ciloxan™, Cipro™, Neofloxin™), levofloxacin (optionally Levaquin™, Tavanic™, Iquix™), norfloxacin (optionally Noroxin™), ofloxacin (optionally Floxin™, Ocuflox™) or gemifloxacin (optionally Factive™);
a macrolide antibiotic,
wherein optionally the macrolide antibiotic is a ketolide antibiotic, clarithromycin (optionally Biaxin™), azithromycin (optionally Zithromax™, Azithrocin™), roxithromycin, erythromycin (optionally Eryc™, Erythrocin™),
and optionally the ketolide antibiotic is telithromycin (Ketek™);
metronidazole (optionally Flagyl™, Metro™, optionally an absorbable metronidazole);
a polyketide antibiotic (optionally anthracimycin, geldanamycin, doxycycline (optionally Doryx™, Doxyhexal™, Doxylin™), erythromycin (optionally Eryc™, Erythrocin™);
rifampicin (also known as rifampin) (optionally Rifadin™),
rifaximin (optionally Xifaxan™, Xifaxanta™, Normix™),
a streptogramin,
wherein optionally the streptogramin is pristinamycin (optionally Pyostacine™), quinupristin, dalfopristin, or both Quinupristin and dalfopristin (optionally Synercid™);
a sulphonamide,
wherein optionally the sulphonamide is hydrochlorothiazide (optionally Apo-hydro™), furosemide (optionally Lasix™) or sulfamethoxazole (optionally Gantanol™);
tinidazole (optionally Fasigyn™, Simplotan™, Tindamax™);
tigecycline (optionally Tygacil™); and
trimethoprim (optionally Proloprim™, Monotrim™, Triprim™).

2. The therapeutic combination, or the composition, of claim 1, wherein the therapeutic drug combination or composition, or individual elements of the therapeutic drug combination or composition, is formulated for administration once a day, b.i.d. (twice a day) or t.i.d, (three times a day), or weekly, or biweekly, or monthly.

3. The therapeutic combination, or the composition, of any of the preceding claims, wherein the therapeutic combination, or the composition, or individual elements of the therapeutic drug combination or composition, are formulated for or formulated as:
administration intravenously, topically, orally, by gargling, by inhalation, by infusion, by injection, by inhalation, intraperitoneally, intramuscularly, subcutaneously, intra-aurally, for intra-articular administration, for intra-mammary administration, for topical administration or for absorption through epithelial or mucocutaneous linings,
conventional or PEGylated liposomal formulations.

4. The therapeutic combination, or the composition, of any of the preceding claims, wherein the therapeutic combination, or the composition, or individual elements of the therapeutic drug combination or composition, is formulated for intermittent or alternated cycling of the drug or active agent or component, and optionally the intermittent or alternated cycling comprises administration weekly, bi-monthly, monthly or quarterly.

5. A pharmaceutical composition or a formulation comprising the therapeutic combination, or a composition, of any of the preceding claims.

6. The pharmaceutical composition or the formulation of any of the preceding claims, further comprising a pharmaceutically acceptable excipient.

7. The pharmaceutical composition or the formulation of any of the preceding claims, wherein the pharmaceutical composition or formulation is formulated or manufactured as a candy, a lollipop (or lollie, pop or sucker), a disposable wafer, strip or patch, a feed, a food, a food or feed concentrate, a pellet, a lozenge, a liquid, a lotion, an implant, a nanoparticle, an elixir, an aerosol, a spray, an inhalant, a powder, a tablet, a pill, a capsule, a gel, a geltab, a nanosuspension, a microparticle or a nanoparticle, a patch, a microgel, a liposome, or a suppository,
and optionally the pharmaceutical composition or the formulation further comprises a probiotic, optionally a probiotic lozenge.

8. A device, a medical device, an implant, a breast implant, a prosthesis, a stent, a catheter, a spray or an aerosol device, an inhaler, a nebulizer, an atomizing device, or a neck, pharyngeal or oral implant: comprising a therapeutic combination, or a composition, of any of the preceding claims, or a pharmaceutical composition or a formulation of any of the preceding claims.

9. A method for: treating, ameliorating or preventing a bacterially-induced disease, condition, infection or reaction in an individual in need thereof; or, applying or administering to a bacterially-affected or a bacterially-infected tissue,
wherein optionally the bacterially-induced condition, disease, infection or reaction is a Streptococcal-induced condition, disease, infection or reaction, or the bacterially-affected or a bacterially-infected tissue comprises a Streptococcal-affected or a Streptococcal-infected tissue,
and optionally the bacterially-induced condition, disease, infection or reaction is located in a tonsil, and optionally the tonsil is a palatine tonsil, an adenoid, and/or a tonsillar tissue at the base of the tongue or next to an eustachian tube,
the method comprising: administering to an individual in need thereof a therapeutic combination or a composition of any of the preceding claims, or a pharmaceutical composition or a formulation of any of the preceding claims, or a device, implant, prosthesis, stent or catheter of any of the preceding claims,
wherein optionally the bacterially-induced condition, disease, infection or reaction in the individual in need thereof is an autoimmune disease, reaction or condition,
wherein optionally the autoimmune disease, reaction or condition is or comprises:
(1) a skin or skin-related autoimmune disease or condition, and optionally the skin or skin-related autoimmune disease or condition is a psoriasis, optionally a plaque-, guttate-, inverse-, pustular- or erythrodermic-type psoriasis; or a Pustulosis Palmaris et Plantaris (PPP) or a Palmoplantar pustulosis,
(6) a Pediatric Autoimmune Neuropsychiatric Disorder (optionally a Pediatric Autoimmune Neuropsychiatric Disorder Associated with a Streptococcal Infection (PANDA)),
(7) rheumatic fever or rheumatic heart disease,
(8) reactive arthritis, or
(9) acute glomerulonephritis,
and optionally the Streptococcal organism comprises a Group A, B, C, D, F, G or H Streptoccocus, a Streptococcus pneumoniae or a Streptoccocus viridans, wherein optionally the Group A Streptococcal organism is a Streptoccocus pyogenes or Streptococcal pyoderma, and optionally the Group B Streptococcal organism is a Streptococcus agalactiae, and optionally the Group F Streptococcal organism is a Streptococcus anginosus (S. anginosus) or a S. salivarius,
and optionally the therapeutic combination or the composition or the pharmaceutical composition or the formulation, or individual elements of the therapeutic combination or the composition or the pharmaceutical composition or the formulation, are administered separately or together, or at the same time, or in synchrony, or by chrono-dosing, or one of the therapeutic agents or drugs is administered before another of the therapeutic agents or drugs,
and optionally when three or more therapeutic combinations or compositions are to be administered to an individual in need thereof during a course of treatment, or when three or more individual active agents (optionally antibiotics) are to be administered to an individual in need thereof during a course of treatment, then two of the three or more of the therapeutic combinations or compositions or three or more of the active agents (optionally antibiotics) are first administered, optionally in one unit dosage form (optionally a tablet, capsule, spray, aerosol or lozenge), and after a period of time (optionally between one week and one month, or between one month and one year) the third or more of the therapeutic combinations or compositions or of the active agents (optionally antibiotics) are added to the treatment regimen to the individual in need thereof,
and optionally the therapeutic combination comprising the three active agents rifabutin, clofazimine and macrolide antibiotic (optionally clarithromycin) is administered to the individual in need thereof, and treatment is initiated by first administering two of the three active agents rifabutin, clofazimine and macrolide antibiotic, and after a period of time (optionally between one week and one month, or between one month and one year) the third active agent antibiotic is added to the treatment regimen to the individual in need thereof, and optionally the first two administered active agents are: rifabutin and clofazimine; rifabutin and macrolide antibiotic; or, clofazimine and macrolide antibiotic,
and optionally when the therapeutic combination comprises the three active agents rifabutin, clofazimine and macrolide antibiotic (optionally clarithromycin) is administered to the individual in need thereof, the rifabutin (optionally Mycobutin™) is administered first at a dosage of between about 100 to 200 mg/day, or at about 150 mg/day (d) (optionally administered in the morning), and about 2 to 4 weeks later the macrolide antibiotic (optionally clarithromycin) is added administered at a dosage of between about 200 to 300 mg/d, or at about 250 mg/d (optionally administered in the morning), and about 2 to 4 weeks later the clofazimine is added at a dose in the range of between about 25 to 150 mg/dose, or 50 to 100 mg/d, or at 75 mg/d (optionally administered in the morning),
and optionally at about 1 week to 2 months, or about 2 to 4 weeks, after commencing of administration of all three rifabutin, clofazimine and macrolide antibiotic, begin administering the same range dosages twice a day, or bid (rifabutin at a dosage of between about 100 to 200 mg/day, the macrolide antibiotic (optionally clarithromycin) is added administered at a dosage of between about 200 to 300 mg/d, the clofazimine is added at a dose in the range of between about 25 to 150 mg/dose, or 50 to 100 mg/d), and optionally the bid dosages of all three rifabutin, clofazimine and macrolide antibiotic remain the same as with once a day dosaging, or the clofazimine bid dosage is set at about 75 mg/d;
and optionally at about 1 week to 2 months, or after about 2 to 4 weeks, after commencing the bid dosaging, increasing the dosage bid of rifabutin and macrolide antibiotic, optionally increasing the dosage bid of rifabutin and macrolide antibiotic to: about 300 mg bid for rifabutin and/or about 500 mg macrolide antibiotic bid, and optionally the clofazimine bid dosage remains the same, or in the range of between about 25 to 150 mg/dose, or 50 to 100 mg/d, or at 75 mg/d,
and optionally the therapeutic combination comprising the three active agents clofazimine, an ansamycin, and amoxicillin is administered to the individual in need thereof, and treatment is initiated by first administering two of the three active agents clofazimine, an ansamycin, and amoxicillin, and after a period of time (optionally between one week and one month, or between one month and one year) the third active agent antibiotic is added to the treatment regimen to the individual in need thereof, and optionally the first two administered active agents are: clofazimine and an ansamycin; an ansamycin and amoxicillin; or clofazimine and amoxicillin,
and optionally the therapeutic combination or the compositions or the pharmaceutical composition or the formulation, or individual elements of the therapeutic combination or the composition or the pharmaceutical composition or the formulation, are formulated for administration intravenously (IV), parenterally, nasally, topically or locally, orally, or by liposome, implant or vessel-targeted nanoparticle delivery,
and optionally further comprises administering a lozenge, a tablet, a liquid, an aerosol, a spray, a geltab or a gel,
wherein optionally the lozenge is a probiotic lozenge, or the tablet, liquid, aerosol, spray, geltab or gel comprises a probiotic,
and optionally the lozenge is a Blis K12 Throat Guard™ or a Blis K12 Throat Guard Boost™ lozenge,
and optionally the probiotic, liquid or gel comprises a non-pathogenic bacterial strain, wherein optionally the non-pathogenic (or attenuated) bacterial strain is or comprises a Streptococcus, optionally a Streptococcus salivarius, optionally Streptococcus salivarius K12,
and optionally the lozenge, liquid or gel is administered after the antibiotics have been administered (after termination of the antibiotic administration regimen),
and optionally the non-pathogenic (or attenuated) bacteria, optionally a Streptococcus strain, partially, substantially or completely replaces (e.g., between about 70% to 90% replaces, between about 80% to 95% replaces, or between about 90% to 99% replaces, or replaces 100%) a pathogenic bacteria in an infected tissue wherein optionally the infected tissue is an adenoid or a tonsil.

10. The method of claim 9, wherein the level of dosing continues daily (or optionally after the second or third daily dose, administered less frequently) for prolonged periods (optionally between one week and ten years), and optionally the dosing can be divided into an induction therapy for about 7 days or 5 days to two weeks, and optionally for up to about four, five or six months, or up to between one month and two years, or two months and one year, with the dose being reduced (optionally halved or quartered, or reduced dosage from between about 10% to about 90% dose reduction) thereafter (optionally after five to 12 days, or after a week, or after two weeks) to a reduced maintenance therapy.

11. The method of claim 9, further comprising before, during or after the applying or administering the therapeutic combination or the compositions or the pharmaceutical composition or the formulation, or individual elements of the therapeutic combination or the composition or the pharmaceutical composition or the formulation:
(a) a surgical procedure comprising a tonsillectomy or adenoid removal, wherein optionally the tonsillectomy is a palatine tonsillectomy and/or an adenoid tonsillectomy, or the method comprises complete or partial removal, freezing, coagulation or ablation of one, any of or all of: a palatine tonsil, an adenoid, and/or a tonsillar tissue at the base of the tongue or next to an eustachian tube; and/or
(b) an immunization of an individual in need thereof against a Streptococcal organism,
wherein optionally the immunization comprises immunizing with an attenuated or a killed pathogenic bacteria, optionally an attenuated or a killed Streptococci, optionally an attenuated or a killed pathogenic Streptococci which has been cultured from a tonsil or a removed tonsil of a patient with a psoriasis,
and optionally the immunization comprises immunizing with a polyvalent immunizing agent,
and optionally the immunization comprises immunizing with an immunizing agent that can either be ingested or used to immunize by injecting into and/or under the skin,
and optionally the immunization is designed to, or results in, an immunized individual developing antibodies and/or a T-cell immunity against a Streptococci (optionally a lymph node Streptococci, optionally a tonsillar lymph node Streptococci),
and optionally the immunization against a Streptococcal organism further comprises administration of an immune-modulator to the individual in need thereof,
and optionally the immune-modulator comprises: alefacept (optionally Amevive™), efalizumab (optionally Raptiva™), etanercept (optionally Enbrel™), ixekinumab (optionally Talz™), golimumab (optionally Simponi™), guselkumab (optionally Tremfya™), cetrolizumab-pegol (optionally Cimzia™), tildrakizumab (optionally Ilumya™), abatacept (optionally Orencia™), an anti-TNF infusion or product (e.g., adalimumab (optionally Humira™, Exemptia™) or infliximab (optionally Remicade™, Remsima™, Inflectra™)), anti-IL 12/23 (optionally ustekinumab, or Stelara™), anti-IL 23, anti-IL 17F, or anti-IL 17A (optionally secukinumab, or Cosentyx™) products, prednisone (optionally Deltasone™, Liquid Pred™, Orasone™, Adasone™, Deltacortisone™, Prednisonum™), cyclosporine or ciclosporine (optionally Neoral™, Sandimmune™), mercaptopurine or 6-MP (6-mercaptopurine) (optionally Purinethol™), methotrexate or amethopterin (optionally Trexall™, Rheumatrex™), tacrolimus (fujimycin) (optionally Prograf™ Advagraf™, Protopic™), ascomycin or immunomycin, rapamycin or sirolimus (optionally Rapamune™), sulfasalazine (optionally Azulfidine™, Salazopyrin™, Sulazine™), a steroid (e.g., a corticosteroid), azathioprine (optionally Azasan™, Imuran™), or a combination thereof,
and optionally administration of the immunomodulator occurs concurrently with the antibiotic administration, optionally administration of the immunomodulator commences as antibiotic treatment commences, or optionally administration of the immunomodulator commences when and if the antibiotic treatment requires support before reaching therapeutically adequate suppression or eradication of the targeted bacteria in the individual (before reaching therapeutically adequate suppression or eradication of the bacteria causing the bacterially-induced disease, condition, infection or reaction in an individual in need thereof, or before reaching therapeutically adequate suppression or eradication of the bacteria infecting the bacterially-affected or a bacterially-infected tissue; and/or
(c) administering a lozenge, a tablet, a liquid, an aerosol, a spray, a geltab or a gel,
wherein optionally the lozenge is a probiotic lozenge, or the tablet, liquid, aerosol, spray, geltab or gel comprises a probiotic,
and optionally the lozenge is a Blis K12 Throat Guard™ or a Blis K12 Throat Guard Boost™ lozenge,
and optionally the probiotic, liquid or gel comprises a non-pathogenic bacterial strain, wherein optionally the non-pathogenic (or attenuated) bacterial strain is or comprises a Streptococcus, optionally a Streptococcus salivarius, optionally Streptococcus salivarius K12,
and optionally the lozenge, liquid or gel is administered after the antibiotics have been administered (after termination of the antibiotic administration regimen),
and optionally the non-pathogenic (or attenuated) bacteria, optionally a Streptococcus strain, partially, substantially or completely replaces (e.g., between about 70% to 90% replaces, between about 80% to 95% replaces, or between about 90% to 99% replaces, or replaces 100%) a pathogenic bacteria in an infected tissue wherein optionally the infected tissue is an adenoid or a tonsil.

12. Use of a therapeutic combination, or a composition, for (or said use being in) the manufacture of a medicament for treating, ameliorating or preventing a bacterially-induced condition, disease, infection or reaction, optionally a Streptococcal-induced condition, disease, infection or reaction, or a bacterial infection, optionally a Streptococcal-Induced or Streptococcal infection, in an individual in need thereof, wherein the therapeutic combination, or the composition comprises a therapeutic combination, or a composition of any of the preceding claims.

13. A formulation or a therapeutic combination for use in a method of treating, ameliorating or preventing a bacterially-induced condition, disease, infection or reaction, optionally a Streptococcal-induced condition, disease, infection or reaction, or a bacterial infection, optionally a Streptococcal-Induced or Streptococcal infection, in an individual in need thereof, wherein the formulation or a therapeutic combination comprises a therapeutic combination, or a composition of any of the preceding claims, and the method comprises a method of any of the preceding claims.
